(19) United States
(12) Patent Application Publication
(10) Pub. No.: US 2010/0189664 A1
(43) Pub. Date: Jul. 29, 2010

(54) COSMETIC AGENTS BASED ON VINYLIMIDAZOLE POLYMERS

(75) Inventors: Ivette G. Castro, Ludwigshafen (DE); Gabi Winter, Wachenheim (DE); Olga Pinneker, Hessheim (DE); Claudia Wood, Weinheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/668,300

(22) PCT Filed: Jul. 7, 2008
(86) PCT No.: PCT/EP2008/058766

§ 371 Date: Jan. 8, 2010

(30) Foreign Application Priority Data
Jul. 9, 2007 (EP) 07112091.9
Jan. 28, 2008 (EP) 08150735.2

(51) Int. Cl. A61K 8/90; A61Q 17/04; A61Q 9/02; A61Q 19/10; A61Q 5/12; A61Q 5/06; A61Q 5/02; A61Q 1/10; A61Q 1/02; C11D 3/37; A61K 8/02

(52) U.S. Cl. 424/52; 424/70.11; 424/78.03; 424/63; 424/59; 424/49; 510/159; 510/119

(57) ABSTRACT

The present invention relates to aqueous or aqueous-alcoholic compositions comprising at least one cationic polymer a) and at least one polymer b) with a molecular weight M_{w} of at most 200 000 g/mol which comprises N-vinylimidazole in copolymerized form, to methods for producing such compositions and to the use of such compositions in cosmetics.

The present invention relates to aqueous or aqueous-alcoholic compositions comprising at least one cationic polymer a) and at least one polymer b) with a molecular weight M_{w} of at most 200 000 g/mol which comprises N-vinylimidazole in copolymerized form, to methods of producing such compositions and to the use of such compositions in cosmetics.

Cationic polymers have already been used for a long time as conditioners in cosmetic formulations. Requirements placed on hair conditioners are, for example, the reduction in the required combing force in wet or dry hair, good detangling upon first combing and good compatibility with cosmetic ingredients customary in conditioners. In addition, cationic polymers reduce the electrostatic charging of hair. The cationic polymers which act as conditioners are usually constituents of shampoos or are provided in separate conditioner preparations.

Usually, the cationic polymers which act as conditioners are provided in shampoos or skin-cleansing compositions together with anionic surfactants.

EP 308189, EP 308190, U.S. Pat. No. 4,803,071 and numerous other patent applications describe conditioners based on copolymers of diallyldimethylammonium chloride (DADMAC) with further monomers.

U.S. Pat. No. 5,275,809 describes the use of ampholytic terpolymers based on DADMAC and 2-acrylamido-2-methylpropanesulfonic acid (AMPS) for haircare.

U.S. Pat. No. 6,110,451 describes conditioners which comprise synergistic combinations of cationic polymers with ampholytic polymers. Polymers comprising vinylimidazole (VI) are not disclosed.

WO 02/083073 describes compositions based on water-soluble polymer-polymer complexes for the treatment of keratin substances. These polymer-polymer complexes consist on the one hand of a water-soluble host polymer and a water-soluble further polymer which is produced in the presence of the host polymer. Polymers comprising vinylimidazole are not described.

EP 1366738 describes cosmetic preparations comprising one or more copolymers of vinylpyrrolidone and vinylimidazole with a cationic charge density of less than or equal to 2 meq/g, one or more guar hydroxypropyltrimethylammonium chlorides, one or more surfactants besides, if appropriate, further cosmetic active ingredients, auxiliaries and additives.

The copolymers of vinylpyrrolidone and vinylimidazole used are of high molecular weight with molecular weights of from 400 000 to 1.8 million, copolymers with a molecular weight of at most 200 000 g/mol are not described.

With numerous cationic polymers from the prior art, incompatibilities arise in the case of typical charge-balancing stoichiometric quantitative ratios of polymer to surfactant. These are evident, for example, from diminishing transparency, i.e. from clouding of the composition which arises.

It was an object of the present invention to provide aqueous compositions in the form of a shampoo or skin-cleansing agent which have very good conditioning effect for hair, skin and nails and at the same time have long-term stability in the presence of anionic surfactants. Furthermore, the compositions should have no build-up effect, be easy to wash out and impart a soft and silky feel to the hair even without the addition of silicones.

These objects are achieved through the provision of an aqueous or aqueous-alcoholic composition comprising

a) at least one cationic polymer a) and

b) at least one polymer b) with a molecular weight M_{w} of at most 200 000 g/mol which comprises N-vinylimidazole in copolymerized form.

Polymer a)

Polymer a) is a cationic polymer. Examples of cationic polymers suitable according to the invention are described in WO 02/083073, paragraph [0054], to which reference is made in its entirety.

Further cationic polymers suitable according to the invention are described in U.S. Pat. No. 6,110,451, column 7, line 46 to column 9, line 38, to which reference is likewise made in its entirety.

Also suitable according to the invention are the polyquaternium-1 to polyquaternium-85 described by the CTFA (Cosmetic, Toiletry, and Fragrance Association, 1101 17th Street, NW Suite 300 Washington, D.C. 20036-4702).

Polymers a) suitable according to the invention are also cationic polyethyleneimines and at least partially hydrolyzed cationic polyvinylcarboxamides, such as, for example, partially hydrolyzed polyvinylformamide.

Polymers a) suitable according to the invention are also homopolymers and copolymers of quaternized dimethylaminoethyl (meth)acrylate (DMAEMA or DMAEA), such as, for example, polyquaternium-37, or of quaternized N,N-dimethyl-aminopropyl(meth)acrylamide (DMAPMAM or DMAPAM), such as, for example, polymethyacrylamidopropyltrimonium chloride. For the at least partial quarternization, dimethyl sulfate or diethyl sulfate are also used.

Also suitable according to the invention are polyvinylpyridinium methochloride, ethyl sulfate or methyl sulfate.

Also suitable according to the invention are polyvinylimidazolium homopolymers or copolymers, such as, for example, polyquaternium-16 (e.g. Luviquat®Excellence, Luviquat®FC 550).

Preferred polymers a) comprise DADMAC in copolymerized form. Preferably, the polymers a) comprise at least 30% by weight, particularly preferably at least 50% by weight, further preferably at least 70% by weight and particularly preferably at least 90% by weight, of DADMAC in copolymerized form.

In one preferred embodiment, polymer a) is a DADMAC homopolymer such as, for example, polyquaternium-6. Polyquaternium-6 is available under the trade names AEC®Polyquaternium-6 (A & E Connock (Perfumery & Cosmetics) Ltd.),

Agequat®400 (CPS Chemical Company), Conditioner®P6 (3V Group), Flocare®C106 (SNF S.A.), Genamin®PDAC (Clariant GmbH), Mackernium®006 (McIntyre Group Ltd), Merquat®100 (Nalco Company), Merquat®106 (Nalco Company), Mirapol®100 (Rhodia Inc.), Octacare®PQ6 (The Associated Octel Company Ltd.), Rheocare®CC6 (Cosmetic Rheologies, Ltd.), Rheocare®CC6P (Cosmetic Rheologies, Ltd.), Ritaquta 6 (Rita Corporation), Salcare®SC30 (Ciba Specialty Chemicals Corporation),

Tinocare®PQ-6H (Ciba Specialty Chemicals Corporation) or Catiofast®CS (BASF). Polymer a) preferably has a molecular weight in the range from 10 000 to 2 million g/mol.

Polymer a preferably has a K value in the range from 20 to 150, preferably from 50 to 120, further preferably from 60 to 100, particularly preferably from 70 to 90 and in particular from 75 to 85.

Preferred polymers a) have, at pH 7, a charge density of at least 2, preferably at least 3, particularly preferably at least 3.5 meq/g. The person skilled in the art knows of methods for determining the charge density of polyelectrolytes, for example the polyelectrolyte titration/streaming potential measurement.

Polymer b)

The water-soluble polymer b) with a molecular weight M_{w} of at most 200 000 g/mol preferably comprises at least 5, further preferably at least 10% by weight and particularly at least 40% by weight of N-vinylimidazole (referred to below as NVI or VI) in copolymerized form. The mass-average molecular weight M_{w} of the water-soluble polymer b) is preferably at most 150 000 g/mol, further preferably at most 120 000 g/mol, in particular at most 90 000 g/mol and is determined by customary measurement methods known to the person skilled in the art. Preferred measurement methods for determining M_{w} are gel permeation chromatography (GPC) and Field Flow Fractionation (FFF). The person skilled in the art knows which measurement conditions are to be used for which polymers.

In one preferred embodiment, the copolymerized NVI is present to at least 80 mol %, particularly preferably to at least 90 mol %, very particularly preferably to at least 95 mol % and in particular to at least 99 mol %, in the nonquaternized state. Most preferably, the copolymerized NVI is present to 100 mol % in the nonquaternized state.

Polymer b) preferably has a K value in the range from 5 to 60, further preferably from 10 to 50, particularly preferably from 20 to 45 and very particularly preferably from 30 to 40.

Polymer b) can comprise further monomers in copolymerized form. Of suitability in principle are all monomers polymerizable with NVI in amounts which are to be chosen so that the resulting polymer b) is water-soluble.

For the purposes of this invention, a polymer is water-soluble if at least 1 g, preferably at least 5 g and particularly preferably at least 10 g, of the polymer dissolve at 20° C. and 1 bar in 1 liter of demineralized water to give a visibly clear solution.

Suitable further monomers are, for example, N-vinylimidazoles of the general formula (I) different from NVI, in which R¹ to R³ is hydrogen, C₁-C₄-alkyl or phenyl

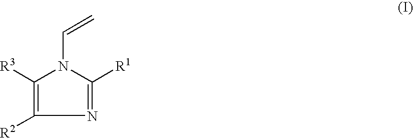

Examples of compounds of the general formula (I) are given in the table below:

| R¹ | R² | R³ |
|---|---|---|
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Also of suitability in principle are, for example, hydrophilic, nonionic compounds, preferably N-vinylamides, N-vinyllactams, esters and amides of subsequently specified α,β-ethylenically unsaturated monocarboxylic acids, vinyl- and allyl-substituted heteroaromatic compounds, esters of α,β-ethylenically unsaturated mono- and dicarboxylic acids with C₁-C₃₀-alkanediols, esters and amides of α,β-ethylenically unsaturated mono- and dicarboxylic acids with C₂-C₃₀-amino alcohols which have a primary or secondary amino group, polyether acrylates and mixtures thereof.

The suitable N-vinyllactams include, for example, N-vinylpyrrolidone, N-vinyl-piperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

N-vinylpyrrolidone is particularly preferably the at least one further monomer during the production of the polymers b).

Suitable monomers for the polymers b) are also primary amides of subsequently specified α,β-ethylenically unsaturated monocarboxylic acids, such as, for example, acrylamide, methacrylamide and ethacrylamide.

Suitable monomers for the polymers b) are also the esters of α,β-ethylenically unsaturated monocarboxylic acids with triols and polyols, such as, for example, glycerol, erythritol, pentaerythritol or sorbitol.

Suitable monomers for the polymers b) may also be α,β-ethylenically unsaturated monocarboxylic acids chosen from the group consisting of acrylic acid, ethacrylic acid, α-chloroacrylic acid, crotonic acid, maleic acid, maleic anhydride, itaconic acid, citraconic acid, mesaconic acid, glutaconic acid, aconitic acid, fumaric acid, half-esters of monoethylenically unsaturated dicarboxylic acids having 4 to 10, preferably 4 to 6, carbon atoms, e.g. of maleic acid, such as monomethyl maleate, and mixtures thereof or vinylsulfonic acid or vinylphosphonic acid or acrylamidoalkanesulfonic acids and salts thereof, such as 2-acrylamido-2-methylpropanesulfonic acid (AMPS). Further suitable monomers for the polymers b) may also be vinyl formate, vinyl acetate, vinyl propionate, vinyl n-butyrate, vinyl stearate, vinyl laurate, styrene, α-methylstyrene, o-chlorostyrene, acrylonitrile, methacrylonitrile, vinyltoluenes, vinyl chloride, vinylidene chloride, vinyl fluoride, vinylidene fluoride, ethylene, propylene, isobutene, butadiene, isoprene, chloroprene, methyl, ethyl, butyl, dodecyl vinyl ethers and mixtures thereof.

Particularly preferred polymers b) comprise at least 15, very particularly preferably at least 20, even more preferably at least 30 and especially preferably at least 40, % by weight of N-vinylimidazole in copolymerized form.

In a further embodiment of the invention, the polymers b) comprise at least 50% by weight, preferred polymers b) comprise at least 70% by weight, further preferred polymers comprise at least 80% by weight and especially preferred polymers comprise at least 90% by weight of N-vinylimidazole in copolymerized form. In a preferred embodiment, polymer b) is a homopolymer of N-vinylimidazole. Apart from N-vinylimidazole, further preferred polymers b) also comprise N-vinyl-pyrrolidone in copolymerized form. Such polymers comprise, for example, at least 20% by weight, preferably at least 30% by weight, further preferably at least 40% by weight and at most 80% by weight, preferably at most 70% by weight, of N-vinyl-pyrrolidone in copolymerized form.

All of the abovementioned polymers with the exception of the vinylimidazole homopolymers can comprise further monomers b3) in copolymerized form, with the proviso that the total amount of all copolymerized monomers is 100% by weight.

Preferred polymers are also polymers b) which comprise

b1) at least 20, preferably at least 25, further preferably at least 30, yet further preferably at least 40, % by weight of N-vinylimidazole,

b2) at least 1, preferably at least 5, further preferably at least 10, particularly preferably at least 20 and at most 80, preferably at most 70, further preferably at most 60, % by weight of N-vinylpyrrolidone

b3) 0 to 49.9% by weight of further monomers in copolymerized form, with the proviso that the sum of b1) to b3) is 100% by weight.

In a particularly preferred embodiment of the invention, polymer b) is a polymer which comprises b1) from 45 to 55% by weight, in particular from 48 to 52% by weight, of N-vinylimidazole, b2) from 55 to 45% by weight, in particular from 52 to 48% by weight, of N-vinylpyrrolidone and b3) from 0 to 10% by weight, in particular from 0 to 4% by weight, of further monomers in copolymerized form, with the proviso that the sum of b1) to b3) is 100% by weight.

One embodiment of the invention is abovementioned compositions where polymer b) has been prepared in the presence of at least one polyether-containing compound b4).

In a preferred embodiment of the invention, the polymers b) are polymerized by polymerization of the respective monomers in the presence of, based on the total amount of 100% by weight of these monomers, 1 to 80% by weight, preferably 5 to 60% by weight, particularly preferably 10 to 50% by weight, of polyether-containing compounds b4).

Further preferred polymers b) for the purposes of this invention are those which are polymerized by polymerization of

b1) at least 20, preferably at least 25, further preferably at least 30, % by weight of N-vinylimidazole,

b2) at least 1, preferably at least 5, further preferably at least 10, particularly preferably at least 20 and at most 80, preferably at most 70, particularly preferably at most 60, % by weight of N-vinylpyrrolidone,

b3) 0 to 50% by weight of further monomers b3) different from b1) and b2), in the presence of

b4) 1 to 80% by weight, preferably 5 to 60% by weight, particularly preferably 10 to 50% by weight, of polyether-containing compounds b4), with the proviso that the sum of b1), b2), b3) is 100% by weight and the amount of b4) is based on the sum of b1), b2) and b3) of 100% by weight.

Particularly suitable polyether-containing compounds b4) have a molecular weight in the range from 300 to 3000 g/mol, preferably from 500 to 2000 g/mol.

Suitable polyether-containing compounds b4) in whose presence the monomers are polymerized are described, for example, in WO 03/046024, p. 4, I. 37 to p. 8, I. 9, to which reference is hereby made in its entirety.

Particularly suitable polyether-containing compounds b4) are polyethylene glycols.

The polymers b) can be prepared in any manner known to the person skilled in the art. Preference is given to free-radical solution polymerization under protective gas at temperatures in the range from 50 to 120° C., preferably from 60 to 90° C., where the initiators used are, for example, water-soluble azo compounds (e.g. 2,2′-azobis-(2-methylpropionamidine)dihydrochloride) and the desired molecular weight is established, for example, through use of so-called regulators.

The weight ratio of polymer a) to polymer b) in the compositions according to the invention is preferably in the range from 10:1 to 1:10, preferably from 5:1 to 1:5, particularly preferably from 3:1 to 1:3 and in particular from 2:1 to 1:2.

This invention further provides a method for producing aqueous or aqueous-alcoholic compositions comprising
a) at least one cationic polymer a) and
b) at least one water-soluble polymer b) with a molecular weight M_{w} of at most 200 000 g/mol, which comprises N-vinylimidazole in copolymerized form,
c) ingredients c) different from a) and b)
   wherein
   the polymers a) and b) are mixed together before or after anionic compounds are added to the composition.

Preference is given to mixing polymers a) and b) together before anionic compounds, in particular anionic surfactants, are added to the composition.

The mixing of the polymers a) and b) can take place at any desired temperatures and pressures on condition that mixing occurs and no notable decomposition of the polymers takes place. Mixing is preferably at temperatures up to 250° C., if appropriate under pressure, and the mixing of polymers a) and b) particularly preferably takes place at temperatures in the range from 10 to 100° C., further preferably in the range from 20 to 90° C. and in particular in the range from 30 to 80° C.

Polymer a) and polymer b) can be mixed together in various ways known to the person skilled in the art. For the mixing, static mixers and also stirring in a reaction container (tank), for example, are highly suitable.

For example, the polymers a) and b) can also be mixed by stirring common solutions, preferably aqueous or aqueous-alcoholic solutions corresponding to the desired quantitative weight ratios. The mixtures can also be prepared by bringing common aqueous or aqueous-alcoholic solutions of the polymers a) and b) to temperatures in the abovementioned ranges and then stirring them further.

Furthermore, it is possible to prepare the mixtures by bringing separate aqueous or aqueous-alcoholic solutions of the polymers a) and b) to temperatures in the abovementioned ranges and then combining the heated solutions and furthermore stirring the resulting common solution at this temperature.

The mixtures according to the invention can, if required for reasons of cosmetic acceptance, be subjected to a physical and/or chemical treatment. Such treatment methods are, for example, the known methods for residual monomer reduction, such as, for example, the addition of one or more polymerization initiators to the mixture at suitable temperatures (for example addition of redox systems such as tert-butyl hydroperoxide/sodium bisulfite) or heating the mixture, a treatment of the polymer mixture by means of steam or stripping with an inert gas, such as with nitrogen, or treatment of the polymer mixture with oxidizing or reducing reagents, adsorption methods such as the adsorption of contamination on selected media such as, for example, activated carbon or an ultrafiltration. It is of course also possible to additionally subject the resulting polymer mixture to an inert-gas and/or steam stripping before or after an afterpolymerization step. Preferably, this stripping operation takes place after the afterpolymerization step. Furthermore, it is possible to largely free the polymer mixture already subjected to an afterpolymerization from residual undesired ingredients which, for example, cause undesired odors, through a vacuum distillation.

In a further preferred embodiment of the invention, the polymer mixtures are prepared by firstly preparing polymer b), reducing the residual amount of monomers (residual monomers), for example by adding a redox system, subjecting the resulting solution of polymer b) to, for example, a vacuum distillation, and then mixing polymer b) with polymer a).

The invention further provides polymer mixtures comprising
a) at least one cationic polymer a) and
b) at least one water-soluble polymer b) with a molecular weight M_{w} of at most 200 000 g/mol which comprises N-vinylimidazole in copolymerized form.

The preferred embodiments of the compositions described above and below also apply, wherever transferable, to the polymer mixtures according to the invention per se.

The present invention further provides a method of producing aqueous or aqueous-alcoholic compositions comprising
a) at least one cationic polymer a) and
b) at least one water-soluble polymer b) with a molecular weight M_{w} of at most 200 000 g/mol which comprises N-vinylimidazole in copolymerized form, wherein
   the polymers a) and b) are mixed together and monomers still present in the mixture are free-radically polymerized.

In a preferred embodiment of the invention, the process step of reducing the monomers still present in the mixture by polymerization is followed still by a further treatment of the composition under reduced pressure. This further treatment is preferably a vacuum distillation.

The quantitative weight ratio of the monomers still present in the mixture to the total amount of the polymers a) and b) is preferably less than 1:100, further preferably less than 1:120, particularly preferably less than 1:150 and in particular less than 1:200.

Aqueous 1% strength by weight solutions of the mixtures of the polymers a) and b) preferably have K values in the range from 60 to 90, preferably from 65 to 80.

The present invention further provides the use of at least one water-soluble polymer b) with a mass-average molecular weight M_{w} of at most 200 000 g/mol for the stabilization of cosmetic preparations which comprise at least one cationic polymer a) as described above and at least one anionic surfactant.

The mass-average molecular weight M_{w} of the water-soluble polymer b) is preferably at most 150 000 g/mol, further preferably at most 120 000 g/mol, in particular at most 90 000 g/mol and is determined by customary measurement methods known to the person skilled in the art. Preferred measurement methods for the determination of M_{w} are gel permeation chromatography (GPC)and Field Flow Fractionation (FFF). The person skilled in the art knows which measurement conditions are to be used for which polymers.

The compositions according to the invention can advantageously be used in cosmetic preparations, in particular hair cosmetic and/or skin cosmetic preparations.

The term cosmetic preparations is to be understood in the wide sense and means all those preparations which are suitable for application to skin and/or hair and/or nails and have a purpose other than a purely medicinal-therapeutic one.

The compositions according to the invention comprising polymer a) and polymer b) are suitable as conditioners in cosmetic preparations, in particular in skin cosmetic and/or hair cosmetic preparations such as shampoos and washing, showering and bath preparations.

The polymers a) and b) together are present in the cosmetic preparations in a fraction of from about 0.001 to 20% by weight, preferably 0.01 to 10% by weight, further preferably 0.01 to 5% by weight and very particularly preferably from 0.05 to 1% by weight, based on the total weight of the agent.

Common aqueous solutions of the polymers a) and b) with a solids content of at most 40% by weight, preferably at most 35% by weight, particularly preferably at most 30% by weight, are largely clear (transmission greater than 90%, preferably greater than 95%, particularly preferably greater than 98%), and are stable during storage at 25° C. for at least 4 weeks, preferably 6 weeks and particularly preferably 3 months. In this connection, stable means that no evidently visible particles are formed and no evidently detectable two-phase or multiphase character forms. The transmission is determined using customary methods known to the person skilled in the art for determining the transmission, such as, for example, photometry.

Hair Cosmetic Preparations and Shampoos

Hair shampoos comprise a large number of different components in order to satisfy the individual requirements placed on the product:

The cleaning power of the shampoos is brought about through the presence of anionic, amphoteric and nonionic surfactants as surface-active compounds in the preparations. Moreover, surfactants provide for the foaming power of the hair-cleansing agents. Important factors when selecting the surfactants are, moreover, their insensitivity toward water hardness, their biodegradability, their compatibility with other components of the preparation, and their price. A widely used shampoo surfactant is, for example, alkyl ether sulfate. Moreover, shampoos comprise a number of consistency regulators which impart the desired viscosity to the preparation. These thickeners bring about an increase in the size of the surfactant micelles and/or swelling of the water phase of the preparation. Thickeners can be chosen from chemically very different classes of substances. Thus, electrolytes (e.g. sodium chloride), alkanolamides (e.g. fatty acid monoethanolamides), fatty alcohols with low degrees of ethoxylation (e.g. diethylene glycol monolauryl ether), highly ethoxylated ethers, esters and diesters, and polymeric thickeners, inter alia, are used. The polymeric thickeners include, for example, cellulose ethers. Moreover, polyacrylates and hydrocolloids can also be used as thickeners. Polymeric thickeners have the great advantage that the viscosity produced by them is largely temperature-independent. Besides perfumes and dyes and a series of compounds which increase the shelf life of the preparations, various types of active ingredients have relatively recently been added to the hair shampoos. Besides UV absorbers, vitamins or plant extracts, these also include so-called hair conditioners, which care for the hair and improve its combability and its feel, and increase its shine. In contrast to most other constituents of shampoos, conditioners attach to the hair and remain there after rinsing. On account of their molecular structure, they position themselves at the damaged site in the cuticula of the hair and smooth the hair. As a result, the hair becomes less rough and brittle, the hairstyle is given considerably more shine and can be combed more easily. The hair also becomes less sensitive to electrostatic charging. Important hair conditioning substances in shampoos are, for example, polymeric quaternary ammonium compounds, cationic cellulose derivatives, polysaccharides or silicone compounds.

The preparations according to the invention lead to a much improved wet and dry combability, and to a significantly improved wet and dry feel of the hair than is the case with conventional mixtures with cationic polymers. Even long hair is barely weighed down, even upon repeated application of the preparation according to the invention, and has a silky shine.

In one preferred embodiment of the invention, besides the polymers a) and b), the shampoos and haircare compositions according to the invention also comprise at least one surfactant.

In one preferred embodiment of the invention, besides the polymers a) and b), the shampoos and haircare agents according to the invention also comprise at least one oil phase and/or fatty phase.

In one preferred embodiment of the invention, besides the polymers a) and b), the shampoos and haircare agents according to the invention also comprise at least one oil phase and/or fatty phase and a surfactant.

Surfactants

Surfactants which can be used are anionic, cationic, nonionic and/or amphoteric surfactants.

Advantageous washing-active anionic surfactants for the purposes of the present invention are acylamino acids and salts thereof, such as
acyl glutamates, in particular sodium acyl glutamate
sarcosinates, for example myristoyl sarcosine, TEA lauroyl sarcosinate, sodium lauroyl sarcosinate and sodium cocoyl sarcosinate,

sulfonic acids and salts thereof, such as
acyl isethionates, for example sodium or ammonium cocoyl isethionate
sulfosuccinates, for example dioctyl sodium sulfosuccinate, disodium laureth sulfosuccinate, disodium lauryl sulfosuccinate and disodium undecylenamido MEA sulfosuccinate, disodium PEG-5 lauryl citrate sulfosuccinate and derivatives, and sulfuric esters, such as
alkyl ether sulfates, for example sodium, ammonium, magnesium, MIPA, TIPA laureth sulfate, sodium myreth sulfate and sodium C₁₂₋₁₃ parethsulfate,
alkyl sulfates, for example sodium, ammonium and TEA lauryl sulfate.

Further advantageous anionic surfactants are
taurates, for example sodium lauroyl taurate and sodium methyl cocoyl taurate,
ethercarboxylic acids, for example sodium laureth-13 carboxylate and sodium PEG-6 cocamide carboxylate, sodium PEG-7 olive oil carboxylate
phosphoric esters and salts, such as, for example, DEA oleth-10 phosphate and dilaureth-4 phosphate,
alkylsulfonates, for example sodium coconut monoglyceride sulfate, sodium C₁₂₋₁₄ olefinsulfonate, sodium lauryl sulfoacetate and magnesium PEG-3 cocamide sulfate,
acyl glutamates, such as di-TEA palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen, and
carboxylic acids and derivatives, such as, for example, lauric acid, aluminum stearate, magnesium alkanolate and zinc undecylenate, ester carboxylic acids, for example calcium stearoyl lactylate, laureth-6 citrate and sodium PEG-4 lauramide carboxylate
alkylarylsulfonates.

The invention provides a composition according to the invention comprising at least one anionic surfactant.

Advantageous washing-active cationic surfactants for the purposes of the present invention are quaternary surfactants. Quaternary surfactants comprise at least one N atom which is covalently bonded to four alkyl or aryl groups. Alkylbetaine, alkylamidopropylbetaine and alkylamidopropyihydroxysultaine, for example, are advantageous.

Further advantageous cationic surfactants for the purposes of the present invention are also
alkylamines,
alkylimidazoles and
ethoxylated amines

and in particular salts thereof.

Advantageous washing-active amphoteric surfactants for the purposes of the present invention are acyl/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut fatty acid amidoethyl-N-hydroxyethyl glycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous washing-active nonionic surfactants for the purposes of the present invention are
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers, alkyl polyglycosides, such as lauryl glucoside, decyl glycoside and cocoglycoside, glycosides with a HLB value of at least 20 (e.g. Belsil®SPG 128V (Wacker)).

Further advantageous nonionic surfactants are alcohols and amine oxides, such as cocamidopropylamine oxide.

Preferred anionic, amphoteric and nonionic shampoo surfactants are mentioned, for example, in “Kosmetik and Hygiene von Kopf bis Fufβ” [Cosmetics and hygiene from head to toe], ed. W. Umbach, 3^{rd} edition, Wiley-VCH, 2004, pp. 131-134, to which reference is made at this point in its entirety.

Among the alkyl ether sulfates, sodium alkyl ether sulfates based on di- or tri-ethoxylated lauryl and myristyl alcohol are particularly preferred. They are significantly superior to the alkyl sulfates with regard to insensitivity toward water hardness, ability to be thickened, low-temperature solubility, and, in particular, skin and mucosa compatibility. They can also be used as the sole washing raw materials for shampoos. Lauryl ether sulfate has better foam properties than myristyl ether sulfate, but is inferior to this with regard to mildness.

Alkyl ether carboxylates with an average and particularly with a higher are generally types of the mildest surfactants, but exhibit poor foaming and viscosity behavior.

They are often used in combination with alkyl ether sulfates and amphoteric surfactants in hair-washing agents.

Sulfosuccinic esters (sulfosuccinates) are mild and readily foaming surfactants, but due to their poor ability to be thickened, are preferably used only together with other anionic and amphoteric surfactants and, due to their low hydrolysis stability, preferably only in neutral or well buffered products.

Amidopropylbetaines are insignificant in practice as sole washing raw materials since their foaming behavior and their ability to be thickened are only moderate. By contrast, these surfactants have excellent skin and eye mucosa compatibility. In combination with anionic surfactants, their mildness can be synergistically improved. Preference is given to the use of cocamidopropylbetaine.

Amphoacetates/amphodiacetates have, as amphoteric surfactants, very good skin and mucosa compatibility and can have a hair-conditioning effect and/or increase the care effect of additives. They are used similarly to the betaines for optimizing alkyl ether sulfate formulations. Sodium cocoamphoacetate and disodium cocoamphodiacetate are most preferred.

Alkyl polyglycosides are nonionic washing raw materials. They are mild, have good universal properties, but are weakly foaming. For this reason, they are preferably used in combinations with anionic surfactants.

Sorbitan esters are likewise types of nonionic washing raw materials. On account of their excellent mildness, they are preferably used for use in baby shampoos. Being weak foamers, they are preferably used in combination with anionic surfactants.

It is advantageous according to the invention if one or more of these surfactants is used in a concentration of from 1 to 30% by weight, preferably in a concentration of from 5 to 25% by weight and very particularly preferably in a concentration of from 10 to 20% by weight, in each case based on the total weight of the composition.

Polysorbates

In addition, polysorbates can be advantageously incorporated according to the invention into the composition as washing-active agents.

Here, polysorbates advantageous for the purposes of the invention are
polyoxyethylene(20) sorbitan monolaurate (Tween 20, CAS No. 9005-64-5)
polyoxyethylene(4) sorbitan monolaurate (Tween 21, CAS No. 9005-64-5)
polyoxyethylene(4) sorbitan monostearate (Tween 61, CAS No. 9005-67-8)
polyoxyethylene(20) sorbitan tristearate (Tween 65, CAS No. 9005-71-4)
polyoxyethylene(20) sorbitan monooleate (Tween 80, CAS No. 9005-65-6)
polyoxyethylene(5) sorbitan monooleate (Tween 81), CAS No. 9005-65-5)
polyoxyethylene(20) sorbitan trioleate (Tween 85, CAS No. 9005-70-3).

In particular,
polyoxyethylene(20) sorbitan monopalmitate (Tween 40, CAS No. 9005-66-7)
polyoxyethylene(20) sorbitan monostearate (Tween 60, CAS No. 9005-67-8)

are particularly advantageous.

According to the invention, these are advantageously used in a concentration of from 0.1 to 5% by weight and in particular in a concentration of from 1.5 to 2.5% by weight, based on the total weight of the composition, individually or as a mixture of two or more polysorbates.

Haircare Agents

It is an aim of haircare to retain the natural state of freshly grown hair over as long a period as possible and, in the case of loss, to restore it. Radiant shine and a pleasant smooth feel are features of natural healthy hair.

For the purposes of this invention, haircare agents are pretreatment agents, hair rinses (hair conditioners, hair balsams), hair treatments, with a distinction being made between treatment products which remain in the hair (leave-on) and those which are rinsed off (rinse-off), hair tonics, styling compositions such as, for example, pomades, styling creams, styling lotions, styling gels (hair gels, wet-look gels, glitter gels), end fluids, hot oil treatments and foam treatments.

Customary formulations of said haircare agents known to the person skilled in the art are given in “Kosmetik and Hygiene von Kopf bis Fuβ” [Cosmetics and hygiene from head to toe], Ed. W. Umbach, 3^{rd} Edition, Wiley-VCH, 2004, Chapter 9.2, pp. 247-264, to which reference is made at this point in its entirety. The ingredients present in the haircare agents besides polymers a) and b) are given above and below and are partly identical to those which may also be present in the abovementioned shampoos according to the invention.

Depending on the field of use, the haircare agents may be applied as spray, foam, gel, gel spray, cream, lotion or wax.

Hairsprays here comprise both aerosol sprays and also pump sprays without propellant gas. Hair foams comprise both aerosol foams and also pump foams without propellant gas. Hairsprays and hair foams comprise preferably predominantly or exclusively water-soluble or water-dispersible components.

If the compounds used in the hairsprays and hair foams according to the invention are water-dispersible, they can be applied in the form of aqueous microdispersions with particle diameters of usually 1 to 350 nm, preferably 1 to 250 nm. The solids contents of these preparations are usually in a range from about 0.5 to 20% by weight. These microdispersions generally require no emulsifiers or surfactants for their stabilization.

Conditioners

Besides the polymers a) and b), the haircare agents and shampoo compositions according to the invention can comprise further conditioners. Conditidners preferred according to the invention are, for example, all compounds which are listed in the International Cosmetic Ingredient Dictionary and Handbook (Volume 4, Editor: R. C. Pepe, J. A. Wenninger, G. N. McEwen, The Cosmetic, Toiletry, and Fragrance Association, 9^{th} Edition, 2002) under Section 4 under the key words Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all of the compounds listed in EP-A 934 956 (pp. 11-13) under “water soluble conditioning agent” and “oil soluble conditioning agent” provided these compounds produce stable preparations with the polymers a) and b) present in the preparations.

Further advantageous conditioning substances are, for example, the compounds different from polymer a) referred to according to INCl as Polyquatemium (in particular Polyquaternium-1 to Polyquaternium-85).

Suitable conditioners include, for example, also polymeric quaternary ammonium compounds, cationic cellulose derivatives, starch derivatives, maltodextrin derivatives and polysaccharide derivatives, as well as quaternary protein hydrolysates and quatemary silicone derivatives.

Conditioners advantageous according to the invention here may be chosen from the compounds shown in Table 1 below.

TABLE 1
Conditioners to be used advantageously

| INCI name | CAS number | Polymer type | Example (trade name) |
|---|---|---|---|
| Polyquaternium-2 | CAS 63451-27-4 | Urea, N,N′-bis[3-(dimethylamino)propyl] | Miranol ® A-15 |
| | | polymer with 1,1′-oxybis(2-chloroethane) | |
| Polyquaternium-5 | CAS 29006-22-4 | Acrylamide, β-methacryloxyethyltriethyl- | |
| | | ammonium methosulfate | |
| Polyquaternium-6 | CAS 26062-79-3 | N,N-dimethyl-N-2-propenyl-2-propen- | Merquat ® 100 |
| | | aminium chloride | |
| Polyquaternium-7 | CAS 26590-05-6 | N,N-dimethyl-N-2-propenyl-2- | Merquat ® S |
| | | propenaminium chloride, 2-propenamide | |
| Polyquaternium-10 | CAS 53568-66-4, | Quaternary ammonium salt of hydroxy- | Celquat ® SC-230M, |
| | 55353-19-0, 54351-50- | ethylcellulose | Polymer JR 400 |
| | 7, 68610-92-4, 81859- | | |
| | 24-7 | | |
| Polyquaternium-11 | CAS 53633-54-8 | Vinylpyrrolidone/dimethylaminoethyl | Gafquat ®755N |
| | | methacrylate copolymer/diethyl sulfate | |
| | | reaction product | |
| Polyquaternium-16 | CAS 29297-55-0 | Vinylpyrrolidone/vinylimideazolinum | Luviquat ® HM552 |
| | | methochloride copolymer | |
| Polyquaternium-17 | CAS 90624-75-2 | | Mirapol ®AD-1 |
| Polyquaternium-19 | CAS 110736-85-1 | Quaternized water-soluble polyvinyl alcohol | |
| Polyquaternium-20 | CAS 110736-86-2 | Quaternized polyvinyl octadecyl ether | |
| | | dispersible in water | |
| Polyquaternium-21 | | Polysiloxane-polydimethyldimethyl- | Abil ® B9905 |
| | | ammonium acetate copolymer | |
| Polyquaternium-22 | CAS 53694-17-0 | Dimethyldiallylammonium chloride/acrylic | Merquat ®280 |
| | | acid copolymer | |
| Polyquaternium-24 | CAS 107987-23-5 | Quaternary ammonium salt of hydroxy- | Quartisoft ® LM-200 |
| | | ethylcellulose | |
| Polyquaternium-28 | CAS 131954-48-8 | Vinylpyrrolidone/methacrylamidopropyltri- | Gafquat ®HS-100 |
| | | methylammonium chloride copolymer | |
| Polyquaternium-29 | CAS 92091-36-6, | Chitosan which has been reacted with | Lexquat ® CH |
| | 48880-30-2 | propylene oxide and quaternized with | |
| | | epichlorohydrin | |
| Polyquaternium-31 | CAS 136505-02-7, | Polymeric, quaternary ammonium salt | Hypan ® QT100 |
| | 139767-67-7 | which is prepared by reacting DMAPA- | |
| | | acrylates/acrylic acid/acrylonitrogen | |
| | | copolymer and diethyl sulfate | |
| Polyquaternium-32 | CAS 35429-19-7 | N,N,N-Trimethyl-2-{[82-methyl-1-oxo-2- | |
| | | propenyl)oxy]ethanaminium chloride, | |
| | | polymer with 2-propenamide | |
| Polyquaternium-37 | CAS 26161-33-1 | | |
| Polyquaternium-44 | | Copolymeric quaternary ammonium salt of | |
| | | vinylpyrrolidone and quaternized | |
| | | imidazoline | |

Further conditioners advantageous according to the invention are cellulose derivatives, in particular Polyquaternium-10 and Polyquatemium-67 (e.g. Ucare® polymer grades, soft-CAT® polymer grades (Dow Chemical)) and quaternized guar gum derivatives, in particular guarhydroxypropylammonium chloride (e.g. Jaguar Excel®, Jaguar C-14S or C-13S, Jaguar C 162® (Rhodia), CAS 65497-29-2, CAS 39421-75-5).

Nonionic poly-N-vinylpyrrolidone/polyvinyl acetate copolymers (e.g. Luviskol® VA 64 (BASF)), anionic acrylate copolymers (e.g. Luviflex® Soft (BASF)), and/or amphoteric amide/acrylate/methacrylate copolymers (e.g. Amphomer® (National Starch)) can also be used advantageously according to the invention as conditioners.

If desired, the conditioners chosen for the cosmetic compositions according to the invention are preferably those conditioners which are described on page 34, line 24 to page 37, line 10 of WO 2006/106140. Reference is hereby made to the content of the cited passage in its entirety.

Thickeners

Thickeners suitable for shampoos are given in “Kosmetik and Hygiene von Kopf bis Fuβ” [Cosmetics and hygiene from head to toe], Ed. W. Umbach, 3^{rd} Edition, Wiley-VCH, 2004, pp. 235-236, to which reference is made at this point in its entirety.

Consistency regulators allow the desired viscosity of shampoos to be established. Thickeners which have a viscosity-building effect by increasing the size of the surfactant micelles and/or through swelling of the water phase originate from chemically very different classes of substance.

Suitable thickeners for the compositions according to the invention are crosslinked polyacrylic acids and derivatives thereof, polysaccharides, such as xanthan gum, guar guar, agar agar, alginates or tyloses, cellulose derivatives, e.g. carboxymethylcellulose or hydroxycarboxymethylcellulose, also higher molecular weight polyethylene glycol mono- and diesters of fatty acids, fatty alcohols, monoglycerides and fatty acids, polyvinyl alcohol and polyvinylpyrrolidone.

Suitable thickeners may, for example, also be chosen advantageously from compounds of the group of gums. The gums include plant or tree saps which harden in the air and form resins or extracts of aquatic plants. For the purposes of the present invention, in this group it is advantageous to choose, for example, gum arabic, carob seed flour, tragacanth, caraya, guar gum, pectin, gellan gum, carrageen, agar, aligns, chondrus, xanthan gum. Also advantageous is the use of derivatized gums such as, for example, hydroxypropyl guar (Jaguar® HP 8).

Among the polysaccharides and polysaccharide derivatives, hyaluronic acid, chitin and chitosan, chondroitin sulfates, starch and starch derivatives, for example, are thickeners advantageous according to the invention.

Among the cellulose derivatives, methylcellulose, carboxymethylcellulose, hydroxylethylcellulose, hydroxypropylmethylcellulose, for example, are advantageous thickeners.

The sheet silicates include naturally occurring and synthetic clay earths, such as, for example, montmorillonite, bentonite, hectorite, laponite, magnesium aluminum silicates such as Veegum®. These can be used as they are or in modified form as thickeners, such as, for example, stearylalkonium hectorites.

In addition, silica gels can also be used advantageously.

The polymers include, for example, polyacrylamides (Seppigel 305), polyvinyl alcohol, polyvinylpyrrolidone, polyvinylpyrrolidone/vinyl acetate copolymers, polyglycols. Suitable thickeners are also polyacrylates, such as Carbopol® (Carbopol® grades 980, 981, 1382, 5984, 2984, EDT 2001, ETD 2020, ETD 2050), Pemulen®TR1 and TR2, Ultrez® (Noveon), Luvigel® EM (BASF), Capigel® 98 (Seppic), Synthalens® (Sigma), the Aculyn® grades from Rohm and Haas, such as Aculyn® 22 (copolymer of acrylates and methacrylic acid ethoxylates with stearyl radical (20 EO units)) and Aculyn® 28 (copolymer of acrylates and methacrylic acid ethoxylates with behenyl radical (25 EO units)).

Suitable thickeners are also, for example, Aerosil grades (hydrophilic silicas), surfactants such as, for example, ethoxylated fatty acid glycerides, esters of fatty acids with polyols such as, for example, pentaerythritol or trimethylolpropane, fatty alcohol ethoxylates with a narrowed homolog distribution or alkyl oligoglucosides, and electrolytes such as sodium chloride and ammonium chloride.

Particularly preferred thickeners for producing gels are Ultrez® 21, Aculyn®28, Luvigel®EM and Capigel®98.

Particularly in the case of more highly concentrated shampoo formulations, it is also possible, to regulate the consistency, to also add substances which reduce the viscosity of the formulation, such as, for example, propylene glycol or glycerol. These substances influence the product properties only slightly.

Preservatives

As products with high water contents, shampoos are preferably protected against the build-up of germs. The most important preservatives used for this purpose are urea condensates, p-hydroxybenzoic esters, the combination of phenoxyethanol with methyldibromoglutaronitrile and acid preservatives with benzoic acid, salicylic acid and sorbic acid.

Shampoo concentrates with high fractions of surfactants or polyols and low water contents can also be formulated without preservatives.

The compositions according to the invention can advantageously comprise one or more preservatives. Advantageous preservatives for the purposes of the present invention are, for example, formaldehyde donors (such as, for example, DMDM hydantoin, which is commercially available, for example, under the trade name Glydant® (Lonza)), iodopropyl butylcarbamates (e.g. Glycacil-L®, Glycacil-S® (Lonza), Dekaben®LMB (Jan Dekker)), parabens (p-hydroxybenzoic alkyl esters, such as, for example, methyl-, ethyl-, propyl- and/or butylparaben), dehydroacetic acid (Euxyl®K 702 (Schülke&Mayr), phenoxyethanol, ethanol, benzoic acid. So-called preservative aids, such as, for example, octoxyglycerol, glycine, soya, etc. are also used advantageously.

The table below gives an overview of customary preservatives:

| | |
|---|---|
| E200 | Sorbic acid |
| E201 | Sodium sorbate |
| E202 | Potassium sorbate |
| E203 | Calcium sorbate |
| E210 | Benzoic acid |
| E211 | Sodium benzoate |
| E212 | Potassium benzoate |
| E213 | Calcium benzoate |
| E214 | Ethyl p-hydroxybenzoate |
| E215 | Ethyl p-hydroxybenzoate Na salt |
| E216 | n-propyl p-hydroxybenzoate |
| E217 | n-propyl p-hydroxybenzoate Na salt |
| E218 | Methyl p-hydroxybenzoate |
| E219 | Methyl p-hydroxybenzoate Na salt |
| E220 | Sulfur dioxide |
| E221 | Sodium sulfite |
| E222 | Sodium hydrogensulfite |
| E223 | Sodium disulfite |
| E224 | Potassium disulfite |
| E226 | Calcium sulfite |
| E227 | Calcium hydrogensulfite |
| E228 | Potassium hydrogensulfite |
| E230 | Biphenyl (diphenyl) |
| E231 | Orthophenylphenol |
| E232 | Sodium orthophenyl phenoxide |
| E233 | Thiabendazole |
| E235 | Natamycin |
| E236 | Formic acid |
| E237 | Sodium formate |
| E238 | Calcium formate |
| E239 | Hexamethylenetetramine |
| E249 | Potassium nitrite |
| E250 | Sodium nitrite |
| E251 | Sodium nitrate |
| E252 | Potassium nitrate |
| E280 | Propionic acid |
| E281 | Sodium propionate |
| E282 | Calcium propionate |
| E283 | Potassium propionate |
| E290 | Carbon dioxide |

Also advantageous are preservatives or preservative aids customary in cosmetics, such as dibromodicyanobutane(2-bromo-2-bromomethylglutarodinitrile), phenoxy-ethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, salicylic acid and salicylates.

It is particularly preferred if the preservatives used are iodopropyl butylcarbamates, parabens (methyl-, ethyl-, propyl- and/or butylparaben) and/or phenoxyethanol.

Preferred preservatives are mixtures of phenoxyethanol, methylparaben, ethylparaben, ethylhexylglycerol and propylene glycol, commercially available as Euxyl® K 350, and mixtures of phenoxyethanol and ethylhexylglycerol, commercially available as Euxyl® PE 9010.

Complexing agents: Since the raw materials and also the shampoos themselves are prepared predominantly in steel apparatuses, the end products can comprise iron (ions) in trace amounts. In order to prevent these impurities adversely affecting the product quality via reactions with dyes and perfume oil constituents, complexing agents such as salts of ethylenediaminetetraacetic acid, of nitrilotriacetic acid, of iminodisuccinic acid or phosphates, are added.

UV photoprotective filters: In order to stabilize the ingredients present in the compositions according to the invention, such as, for example, dyes and perfume oils, against changes due to UV light, it is possible to incorporate UV photoprotective filters, such as, for example, benzophenone derivatives. Of suitability for this are all cosmetically acceptable UV photoprotective filters.

Antioxidants: An additional content of antioxidants is generally preferred. According to the invention, antioxidants which can be used are all antioxidants which are customary or suitable for cosmetic and/or dermatological applications. The antioxidants are advantageously chosen from the group consisting of amino acids (e.g. glycine, histidine, tyrosine, trytophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodopropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives therof, α-glycosylrutin, ferulic acid, furfurylideneglucitol, carnosine, butylhydroxytoluene, butylhydroxyanisole, nordihydroguaiacic resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the abovementioned antioxidants (one or more compounds) in the compositions is preferably 0.001 to 30% by weight, particularly preferably 0.05 to 20% by weight, in particular 0.1 to 10% by weight, based on the total weight of the composition.

If vitamin E and/or derivatives thereof are the antioxidant or the antioxidants, it is advantageous to provide these in concentrations of from 0.001 to 10% by weight, based on the total weight of the composition.

If vitamin A, or vitamin A derivatives, or carotenes or derivatives thereof are the antioxidant or the antioxidants, it is advantageous to provide these in concentrations of from 0.001 to 10% by weight, based on the total weight of the composition.

Buffers: Buffers ensure the pH stability of the shampoo. Citrate, lactate and phosphate buffers are primarily used.

Solubility promoters: These are used in order to form clear solutions of care oils or perfume oils and also to keep them in clear solution at low temperatures. The most common solubility promoters are ethoxylated nonionic surfactants, e.g. hydrogenated and ethoxylated castor oils.

Antimicrobial agents: In addition, it is also possible to use antimicrobial agents. These include generally all suitable preservatives with a specific action against Gram-positive bacteria, e.g. triclosan (2,4,4′-trichloro-2′-hydroxydiphenyl ether), chlorhexidene (1,1′-hexamethylenebis[5-(4-chlorophenyl)biguanide), and TTC (3,4,4′-trichlorocarbanilide). Quaternary ammonium compounds are in principle likewise suitable and are preferably used for disinfectant soaps and washing lotions. Numerous fragrances also have antimicrobial properties. A large number of essential oils or their characteristic ingredients, such as, for example, oil of cloves (eugenol), mint oil (menthol) or thyme oil (thymol), also exhibit marked antimicrobial effectiveness.

The antimicrobially effective substances are generally used in concentrations of from about 0.1 to 0.3% by weight.

Dispersants: If it is the aim to disperse insoluble active ingredients, e.g. antidandruff active ingredients or silicone oils, in the shampoo and keep them permanently in suspension, it is advantageous to use dispersants and thickeners, such as, for example, magnesium aluminum silicates, bentonites, fatty acyl derivatives, polyvinylpyrrolidone or hydrocolloids, e.g. xanthan gum or carbomers.

According to the invention, preservatives are present in a total concentration of at most 2% by weight, preferably at most 1.5% by weight and particularly preferably at most 1% by weight, based on the total weight of the composition.

Oils, Fats and Waxes

In addition, the compositions according to the invention comprise, in one embodiment of the invention, oils, fats or waxes.

Constituents of the oil phase and/or fatty phase of the composition according to the invention are advantageously chosen from the group of lecithins and of fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, be chosen advantageously from the group of synthetic, semisynthetic and natural oils, such as, for example, olive oil, sunflower oil, soya oil, peanut oil, rapeseed oil, almond oil, palm oil, coconut oil, castor oil, wheatgerm oil, grapeseed oil, thistle oil, evening primrose oil, macadamia nut oil and the like. Further polar oil components can be chosen from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, and from the group of esters of aromatic carboxylic acids and saturated and/or unsaturated, branched and/or unbranched alcohols of chain length from 3 to 30 carbon atoms. Such ester oils can then advantageously be chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (myritol 331), butylene glycol dicaprylate/dicaprate and dibutyl adipate, and synthetic, semisynthetic and natural mixtures of such esters, such as, for example, jojoba oil.

In addition, one or more oil components can advantageously be chosen from the group of branched and unbranched hydrocarbons and hydrocarbon waxes, silicone oils, dialkyl ethers, the group of saturated or unsaturated, branched or unbranched alcohols.

Any mixtures of such oil and wax components are also to be used advantageously for the purposes of the present invention. If appropriate, it may also be advantageous to use waxes, for example cetyl palmitate, as the sole lipid component of the oil phase.

According to the invention, the oil component is advantageously chosen from the group 2-ethylhexyl isostearate, octyldodecanol, isotridecyl isononanoate, isoeicosane, 2-ethylhexyl cocoate, C12-15-alkyl benzoate, caprylic/capric triglyceride, dicaprylyl ether.

According to the invention, mixtures of C12-15-alkyl benzoate and 2-ethylhexyl isostearate, mixtures of C12-15-alkyl benzoate and isotridecyl isononanoate, and mixtures of C12-15-alkyl benzoate, 2-ethylhexyl isostearate and isotridecyl isononanoate are advantageous.

According to the invention, fatty acid triglycerides, in particular soya oil and/or almond oil, are particularly preferably used as oils with a polarity of from 5 to 50 mN/m.

Of the hydrocarbons, paraffin oil, squalane, squalene and in particular (if appropriate hydrogenated) polyisobutenes are to be used advantageously for the purposes of the present invention.

In addition, the oil phase can be chosen advantageously from the group of Guerbet alcohols. They are formed according to the reaction equation

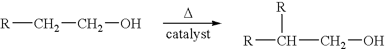

by oxidation of an alcohol to give an aldehyde, by aldol condensation of the aldehyde, elimination of water from the aldol and hydrogenation of the allyl aldehyde. Guerbet alcohols are liquid even at low temperatures and cause virtually no skin irritations. They can be used advantageously as fatting, superfatting and also refatting constituents in cosmetic compositions.

The use of Guerbet alcohols in cosmetics is known per se. Such species are then mostly characterized by the structure

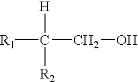

Here, R₁ and R₂ are generally unbranched alkyl radicals.

According to the invention, the Guerbet alcohol or alcohols are advantageously chosen from the group where

R₁=propyl, butyl, pentyl, hexyl, heptyl or octyl and

R₂=hexyl, heptyl, octyl, nonyl, decyl, undecyl, dodecyl, tridecyl or tetradecyl.

Guerbet alcohols preferred according to the invention are 2-butyloctanol (commercially available, for example, as Isofol® 12 (Condea)) and 2-hexyldecanol (commercially available, for example, as Isofol® 16 (Condea)).

Mixtures of Guerbet alcohols according to the invention are also to be used advantageously according to the invention, such as, for example, mixtures of 2-butyl-octanol and 2-hexyldecanol (commercially available, for example, as Isofol® 14 (Condea)).

Any mixtures of such oil and wax components are also to be used advantageously for the purposes of the present invention.

The oil component can also advantageously have a content of cyclic or linear or silicone oils or consist entirely of such oils, although it is preferred to use an additional content of other oil phase components apart from the silicone oil or the silicone oils.

Low molecular weight silicones or silicone oils are generally defined by the following general formula

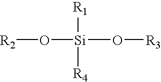

Higher molecular weight silicones or silicone oils are generally defined by the following general formula

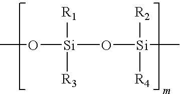

where the silicon atoms may be substituted by identical or different alkyl radicals and/or aryl radicals, which are represented here in general terms by the radicals R₁ to R₄. However, the number of different radicals is not necessarily restricted to 4. m can here assume values from 2 to 200 000. Cyclic silicones to be used advantageously according to the invention are generally defined by the following general formula

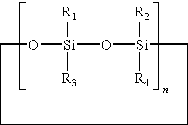

where the silicon atoms can be substituted by identical or different alkyl radicals and/or aryl radicals, which are shown here in general terms by the radicals R₁ to R₄. However, the number of different radicals is not necessarily restricted to 4. n can here assume values from 3/2 to 20. Fractional values for n take into account that uneven numbers of siloxyl groups may be present in the cycle.

Phenyltrimethicone is advantageously chosen as silicone oil. Other silicone oils, for example dimethicone, hexamethylcyclotrisiloxane, phenyldimethicone, cyclomethicone (e.g. decamethylcyclopentasiloxane), hexamethylcyclotrisiloxane, polydimethylsiloxane, poly(methylphenylsiloxane), cetyldimethicone, behenoxydimethicone are also to be used advantageously for the purposes of the present invention. Also advantageous are mixtures of cyclomethicone and isotridecyl isononanoate, and those of cyclomethicone and 2-ethylhexyl isostearate.

It is, however, also advantageous to choose silicone oils of similar constitution to the compounds described above whose organic side chains are derivatized, for example polyethoxylated and/or polypropoxylated. These include, for example, polysiloxane polyalkyl-polyether copolymers, such as, for example, cetyldimethicone copolyol. Cyclomethicone (octamethylcyclotetrasiloxane) is used advantageously as silicone oil to be used according to the invention.

Fat and/or wax components to be used advantageously according to the invention can be chosen from the group of vegetable waxes, animal waxes, mineral waxes and petrochemical waxes. For example, candelilla wax, carnauba wax, Japan wax, espartograss wax, cork wax, guaruma wax, ricegerm oil wax, sugarcane wax, berry wax, ouricury wax, montan wax, jojoba wax, shea butter, beeswax, shellac wax, spermaceti, lanolin (wool wax), europygial grease, ceresin, ozokerite (earth wax), paraffin waxes and microwaxes are advantageous.

Further advantageous fat and/or wax components are chemically modified waxes and synthetic waxes, such as, for example, Synchrowax® HRC (glyceryl tribehenate), and Syncrowax® AW 1 C (C₁₈₋₃₆-fatty acid), and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkyl hydroxystearoylstearate and/or glycol montanate. Further advantageous are also certain organosilicon compounds which have similar physical properties to the specified fat and/or wax components, such as, for example, stearoxytrimethylsilane.

According to the invention, the fat and/or wax components can be used either individually or as a mixture in the composition.

Any mixtures of such oil and wax components are also to be used advantageously for the purposes of the present invention.

The oil phase is advantageously chosen from the group 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric triglyceride, dicaprylyl ether. Of-particular advantage are mixtures of octyldodecanol, caprylic/capric triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and mixtures of C₁₂₋₁₅-alkyl benzoate, 2-ethylhexyl isostearate and isotridecyl isononanoate.

Of the hydrocarbons, paraffin oil, cycloparaffin, squalane, squalene, hydrogenated polyisobutene and polydecene are to be used advantageously for the purposes of the present invention.

The oil component is also advantageously chosen from the group of phospholipids. The phospholipids are phosphoric esters of acylated glycerols. Of greatest importance among the phosphatidylcholines are, for example, the lecithins, which are characterized by the general structure

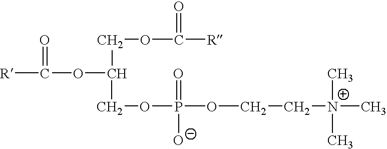

where R′ and R″ are typically unbranched aliphatic radicals having 15 or 17 carbon atoms and up to 4 cis double bonds. Paraffin oil advantageous according to the invention which can be used in accordance with the invention is Merkur Weissoel Pharma 40 from Merkur Vaseline, Shell Ondina® 917, Shell Ondina® 927, Shell Oil 4222, Shell Ondina® 933 from Shell & DEA Oil, Pionier® 6301 S, Pionier® 2071 (Hansen & Rosenthal).

Suitable cosmetically compatible oil and fat components are described in Karl-Heinz Schrader, Grundlagen und Rezepturen der Kosmetika [Fundamentals and formulations of cosmetics], 2^{nd} Edition, Verlag Hüthig, Heidelberg, pp. 319-355, to which reference is hereby made in its entirety.

The content of oils, fats and waxes is at most 50% by weight, preferably 30% by weight, further preferably at most 20% by weight, based on the total weight of the composition.

According to the invention, apart from the abovementioned substances, the compositions comprise, if appropriate, the additives customary in cosmetics or dermatology, for example perfume, dyes, antimicrobial substances, refatting agents, complexing and sequestering agents, pearlizing agents, plant extracts, vitamins, active ingredients, preservatives, bactericides, pigments which have a coloring effect, thickeners, softening, moisturizing and/or humectant substances, or other customary constituents of a cosmetic or dermatological formulation, such as alcohols, polyols, polymers, organic acids for adjusting the pH, foam stabilizers, electrolytes, organic solvents or silicone derivatives.

With regard to the specified further ingredients known to the person skilled in the art for the compositions, reference may be made to “Kosmetik und Hygiene von Kopf bis Fuβ” [Cosmetics and hygiene from head to toe], Ed. W. Umbach, 3^{rd} Edition, Wiley-VCH, 2004, pp. 123-128, to which reference is made at this point in its entirety.

Ethoxylated Glycerol Fatty Acid Esters

The shampoos and haircare agents according to the invention comprise, if appropriate, ethoxylated oils chosen from the group of ethoxylated glycerol fatty acid esters, particularly preferably PEG-10 olive oil glycerides, PEG-11 avocado oil glycerides, PEG-11 coco butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glycerol cocoate, PEG-45 palm kernel oil glycerides, PEG-35 castor oil, olive oil-PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride-PEG-6 ester, PEG-20 corn oil glycerides, PEG-18 glyceryl oleate-cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are PEG-7 glyceryl cocoate, PEG-9 cocoglycerides, PEG-40 hydrogenated castor oil, PEG-200 hydrogenated glyceryl palmate.

Ethoxylated glycerol fatty acid esters are used in aqueous cleaning formulations for various purposes. Glycerol fatty acid esters with a degree of ethoxylation of about 30-50 serve as solubility promoters for nonpolar substances such as perfume oils. Highly ethoxylated glycerol fatty acid esters are used as thickeners.

Active Ingredients

It has been found that a very wide variety of active ingredients of varying solubility can be incorporated homogeneously into the shampoos and haircare agents according to the invention. The substantivity of the active ingredients on the hair is higher from the described composition than from conventional surfactant-containing cleaning formulations.

According to the invention, the active ingredients (one or more compounds) can advantageously be chosen from the group consisting of acetylsalicylic acid, atropine, azulene, hydrocortisone and derivatives thereof, e.g. hydrocortisone-17 valerate, vitamins of the B and D series, in particular vitamin B1, vitamin B12, vitamin D, vitamin A and derivatives thereof, such as retinyl palmitate, vitamin E or derivatives thereof, such as, for example, tocopheryl acetate, vitamin C and derivatives thereof, such as, for example, ascorbyl glucoside, but also niacinamide, panthenol, bisabolol, polydocanol, unsaturated fatty acids, such as, for example, the essential fatty acids (usually referred to as vitamin F), in particular γ-linolenic acid, oleic acid, eicosapentanoic acid, docosahexanoic acid and derivatives thereof, chloramphenicol, caffeine, prostaglandins, thymol, camphor, squalene, extracts or other products of vegetable and animal origin, e.g. evening primrose oil, borage oil or blackcurrant seed oil, fish oils, cod liver oil, and also ceramides and ceramide-like compounds, incense extract, green tea extract, water lily extract, licorice extract, hamamelis, antidandruff active ingredients (e.g. selenium disulfide, zinc pyrithione, piroctone, olamine, climbazole, octopirox, polydocanol and combinations thereof), complexing active ingredients, such as, for example, those comprising γ-oryzanol and calcium salts such as calcium pantothenate, calcium chloride, calcium acetate. It is also advantageous to choose the active ingredients from the group of refatting substances, for example purcellin oil, Eucerit® and Neocen®.

The active ingredient or ingredients is/are particularly advantageously chosen from the group of NO synthase inhibitors, particularly if the compositions according to the invention are to be used for the treatment and prophylaxis of the symptoms of intrinsic and/or extrinsic ageing, and for the treatment and prophylaxis of the harmful effects of ultraviolet radiation on the hair. A preferred NO synthase inhibitor is nitroarginine.

The active ingredient or ingredients are further advantageously chosen from the group comprising catechins and bile esters of catechins and aqueous or organic extracts from plants or parts of plants which have a content of catechin or bile esters of catechins, such as, for example, the leaves of the Theaceae family, in particular the Camellia sinensis (green tea) species. Of particular advantage are their typical ingredients (e.g. polyphenols and catechins, caffeine, vitamins, sugars, minerals, amino acids, lipids).

Catechins are a group of compounds which are to be regarded as hydrogenated flavones or anthocyanidins and are derivatives of “catechin” (catechol, 3,3′,4′,5,7-flavanepentaol, 2-(3,4-dihydroxyphenyl)chroman-3,5,7-triol). Epicatchin ((2R,3R)-3,3′,4′,5,7-flavanepentaol) is also an advantageous active ingredient for the purposes of the present invention.

Also advantageous are plant extracts with a content of catechins, in particular extracts of green tea, such as, for example, extracts from leaves of the plants of the Camellia spec. species, very particularly of the tea varieties Camellia sinensis, C. assamica, C. taliensis and C. inawadiensis and hybrids of these with, for example, Camellia japonica.

Preferred active ingredients are also polyphenols and catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (often also collectively called “flavones”) are also advantageous active ingredients for the purposes of the present invention. They are characterized by the following basic structure (substitution positions given):

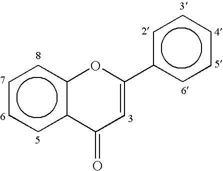

Some of the more important flavones which can also preferably be used in compositions according to the invention are listed in table 2 below.

TABLE 2
Table 2: Flavones

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones generally occur in glycosylated form.

According to the invention, the flavonoids are preferably chosen from the group of substances of the general formula

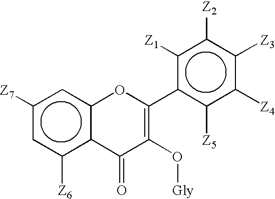

where Z₁ to Z₇, independently of one another, are chosen from the group H, OH, alkoxy and hydroxyalkoxy, where the alkoxy and/or hydroxyalkoxy groups may be branched or unbranched and have 1 to 18 carbon atoms, and where Gly is chosen from the group of mono- and oligoglycoside radicals.

According to the invention, the flavonoids can, however, also be chosen advantageously from the group of substances of the general formula

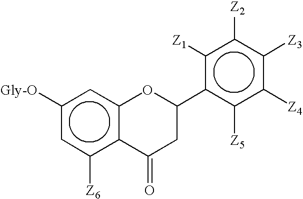

where Z₁ to Z₆, independently of one another, are chosen from the group H, OH, alkoxy and hydroxyalkoxy, where the alkoxy and/or hydroxyalkoxy groups may be branched or unbranched and have 1 to 18 carbon atoms, and where Gly is chosen from the group of mono- and oligoglycoside radicals.

Preferably, such structures can be chosen from the group of substances of the general formula

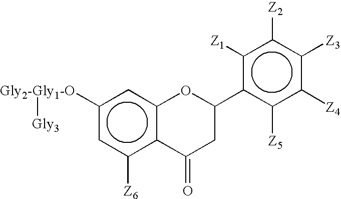

where Z₁ to Z₈, independently of one another, are as specified above and Gly₁, Gly₂ and Gly₃, independently of one another, are monoglycoside radicals or oligoglycoside radicals. Gly₂ and Gly₃ can also individually or together be saturations by hydrogen atoms.

Preferably, Gly₁, Gly₂ and Gly₃, independently of one another, are chosen from the group of hexosyl radicals, in particular rhamnosyl radicals and glucosyl radicals. However, other hexosyl radicals, for example allosyl, altrosyl, galactosyl, gulosyl, idosyl, mannosyl and talosyl are also to be used advantageously, if appropriate. According to the invention, it may also be advantageous to use pentosyl radicals.

Advantageously, Z₁ to Z₅, independently of one another, are chosen from the group H, OH, methoxy, ethoxy and 2-hydroxyethoxy, and the flavone glycosides correspond to the general structural formula

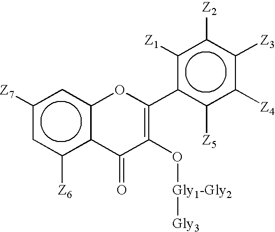

The flavone glycosides are particularly advantageously chosen from the group which is given by the following structure,

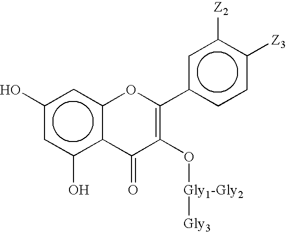

where Gly₁, Gly₂ and Gly₃, independently of one another, are monoglycoside radicals or oligoglycoside radicals. Gly₂ and Gly₃ can also individually or together be saturations by hydrogen atoms.

Preferably, Gly₁, Gly₂ and Gly₃, independently of one another, are chosen from the group of hexosyl radicals, in particular rhamnosyl radicals and glucosyl radicals. However, other hexosyl radicals, for example, allosyl, altrosyl, galactosyl, gulosyl, idosyl, mannosyl and talosyl, are also to be used advantageously, if appropriate. According to the invention, it may also be advantageous to use pentosyl radicals.

For the purposes of the present invention, it is particularly advantageous to choose the flavone glycoside or flavone glycosides from the group α-glucosylrutin, α-glucosylmyricetin, α-glucosylisoquercitrin, α-glucosylisoquercetin and α-glucosyl-quercitrin.

Further advantageous active ingredients are sericoside, pyridoxol, vitamin K, biotin and aroma substances.

Furthermore, the active ingredients (one or more compounds) can also very advantageously be chosen from the group of hydrophilic active ingredients, in particular from the following group:

α-hydroxy acids, such as lactic acid or salicylic acid and salts thereof, such as, for example, Na lactate, Ca lactate, TEA lactate, urea, allantoin, serine, sorbitol, glycerol, milk proteins, panthenol, chitosan.

The list of specified active ingredients and active ingredient combinations which can be used in the compositions according to the invention is not of course intended to be limiting. The active ingredients can be used individually or in any combinations with one another.

The amount of such active ingredients (one or more compounds) in the compositions according to the invention is preferably 0.001 to 30% by weight, particularly preferably 0.05 to 20% by weight, in particular 1 to 10% by weight, based on the total weight of the composition.

The specified and further active ingredients which can be used in the compositions according to the invention are given in DE 103 18 526 A1 on pages 12 to 17, to which reference is hereby made at this point in its entirety.

Pearlizing Agents

Advantageous embodiments of the cosmetic compositions according to the invention are also characterized in that they comprise opacifiers and/or pearlizing agents as further constituents. Opacifiers are understood according to the invention as meaning substances and mixtures of substances which impart a cloudy emulsion-like appearance to the preparation. Pearlizing agents are understood here according to the invention as meaning substances or mixtures of substances which impart an opalescent appearance to the preparation. According to the invention, it is also advantageous to use mixtures of opacifiers and pearlizing agents.

Opacifiers/pearlizing agent and mixtures advantageous according to the invention are, inter alia:
PEG-3 distearate (e.g. CUTINA®TS from Cognis),
a combination of glycol distearate, glycerol, laureth-4 and cocamidopropylbetaine (e.g. EuperlaePK3000 and Euperlan®PK4000 from Cognis),
a combination of glycol distearate, cocoglucosides, glyceryl oleate and glyceryl stearate (e.g. Lamesoft®Benz from Cognis).
Styrene/acrylate copolymers (e.g. Acusol®OP 301 from Rohm & Haas)

Suitable pearlescent waxes are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxyl-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates which have a total of at least 24 carbon atoms, specifically laurone and distearyl ether; fatty acids such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 11 hydroxyl groups, and mixtures thereof.

The compositions according to the invention can also comprise glitter substances and/or other effect substances (e.g. color streaks).

Emulsifiers

In one preferred embodiment, the shampoos and haircare agents according to the invention additionally comprise emulsifiers. Suitable emulsifiers are, for example, nonionogenic surfactants from at least one of the following groups:
(1) addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
(2) C12/18 fatty acid mono- and diesters of addition products of from 1 to 30 mol of ethylene oxide onto glycerol;
(3) glycerol mono- and diesters and sorbitan mono- and diesters of saturated and unsaturated fatty acids having 6 to 22 carbon atoms and ethylene oxide addition products thereof;
(4) alkyl mono- and oligoglycosides having 8 to 22 carbon atoms in the alkyl radical and ethoxylated analogues thereof;
(5) addition products of from 15 to 60 mol of ethylene oxide onto oils, for example onto castor oil and/or hydrogenated castor oil;
(6) polyol, and in particular polyglycerol, esters, such as, for example, polyglycerol polyricinoleate, polyglycerol poly-12-hydroxystearate or polyglycerol dimerate. Mixtures of compounds from two or more of these classes of substances are likewise suitable;
(7) addition products of from 2 to 15 mol of ethylene oxide onto castor oil and/or hydrogenated castor oil;
(8) partial esters based on linear, branched, unsaturated or saturated C_{6/22}-fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose);
(9) mono-, di- and trialkyl phosphates, and mono-, di- and/or tri-PEG alkyl phosphates and salts thereof;
(10) wool wax alcohols;
(11) polysiloxane-polyalkyl-polyether copolymers and corresponding derivatives;
(12) mixed esters of pentaerythritol, fatty acids, citric acid and fatty alcohols according to DE-C 1165574 and/or mixed esters of fatty acids having 6 to 22 carbon atoms, methylglycose and polyols, preferably glycerol or polyglycerol and
(13) polyalkylene glycols.

The addition products of ethylene oxide and/or of propylene oxide onto fatty alcohols, fatty acids, alkylphenols, glycerol mono- and diesters, and sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C₁₂ to C₁₈-fatty acid mono- and diesters of addition products of ethylene oxide onto glycerol are known from DE-C 2024051 as refatting agents for cosmetic preparations. C₈ to C₁₈-alkyl mono- and oligoglycosides, their preparation and their use are known from the prior art.

They are prepared in particular by reacting glucose or oligosaccharides with primary alcohols having 8 to 18 carbon atoms. As regards the glycoside ester, both monoglycosides in which one cyclic sugar radical is bonded glycosidically to the fatty alcohol, and also oligomeric glycosides with a degree of oligomerization up to preferably about 8 are suitable. The degree of oligomerization here is a statistical average value which is based on a homolog distribution customary for such technical products.

In addition, zwitterionic surfactants can be used as emulsifiers. Zwitterionic surfactants is the term used to describe those surface-active compounds which carry at least one quaternary ammonium group and at least one carboxylate and/or one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethyl-ammonium glycinates, for example cocoacylaminopropyldimethylammonium glycinate, and 2-alkyl-3-carboxymethyl-3-hydroxyethylimidazolines each having 8 to 18 carbon atoms in the alkyl or acyl group, and cocoacylaminoethyl hydroxyethyl-carboxymethyl glycinate.

Of particular preference is the fatty acid amide derivative known under the CTFA name Cocamidopropyl Betaine. Likewise suitable emulsifiers are ampholytic surfactants. Ampholytic surfactants are understood as meaning those surface-active compounds which, apart from a C₈ to C₁₈-alkyl or -acyl group in the molecule, comprise at least one free amino group and at least one —COOH and/or —SO₃H group and are capable of forming internal salts. Examples of suitable ampholytic surfactants are N-alkylglycines, N-alkylpropionic acids, N-alkylaminobutyric acids, N-alkylaminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂ to C₁₈-acylsarcosine.

Besides the ampholytic emulsifiers, quaternary emulsifiers are also suitable, particular preference being given to those of the ester quat type, preferably methyl-quaternized difatty acid triethanolamine ester salts.

Perfume Oils

If appropriate, the shampoos and haircare agents according to the invention can comprise perfume oils. Perfume oils which may be mentioned are, for example, mixtures of natural and synthetic fragrances. Natural fragrances are extracts from flowers (lily, lavender, rose, jasmine, neroli, ylang-ylang), stems and leaves (geranium, patchouli, petitgrain), fruits (anise, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, cibet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, 4-tert-butyl cyclohexylacetate, linoleyl acetate, dimethylbenzylcarbonyl acetate, phenylethyl acetate, linoleyl benzoate, benzyl formate, ethylmethyl phenylglycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonat, the ketones include, for example, the ionones, cc-isomethylionine and methyl cedryl ketone, the alcohols include anethol, citronnel, eugenol, isoeugenol, geraniol, linalool, phenylethyl alcohol and terioneol, and the hydrocarbons include primarily the terpenes and balsams. However, preference is given to using mixtures of different fragrances which together produce a pleasing scent note. Essential oils of lower volatility, which are mostly used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, Boisambrene®Forte, ambroxan, indole, hedione, sandelice, citrus oil, mandarin oil, orange oil, allyl amyl glycolate, cyclovertal, lavandin oil, clarry sage oil, β-damascone, geranium oil bourbon, cyclohexyl salicylate, Vertofix®Coeur, Iso-E-Super®, Fixolid®NP, evernyl, iraldein gamma, phenylacetic acid, geranyl acetate, benzyl acetate, rose oxide, romillat, irotyl and floramat alone or in mixtures.

Pigments

If appropriate, the shampoos and haircare agents according to the invention further comprise pigments.

The pigments are present in the product mass in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm. The pigments are colorants which are virtually insoluble in the application medium and may be inorganic or organic. Inorganic-organic mixed pigments are also possible. Preference is given to inorganic pigments. The advantage of the inorganic pigments is their excellent fastness to light, weather and temperature. The inorganic pigments may be of natural origin, for example prepared from chalk, ocher, umber, green earth, burnt sienna or graphite. The pigments may be white pigments, such as, for example, titanium dioxide or zinc oxide, black pigments, such as, for example, iron oxide black, chromatic pigments, such as, for example, ultramarine or iron oxide red, luster pigments, metal effect pigments, pearlescent pigments, and fluorescent or phosphorescent pigments, where preferably at least one pigment is a colored, nonwhite pigment.

Metal oxides, hydroxides and oxide hydrates, mixed phase pigments, sulfur-containing silicates, metal sulfides, complex metal cyanides, metal sulfates, chromates and molybdates, and the metals themselves (bronze pigments) are suitable. Of particular suitability are titanium dioxide (CI77891), black iron oxide (CI77499), yellow iron oxide (CI77490), red and brown iron oxide (CI77491), manganese violet (CI77742), ultramarine (sodium aluminum sulfosilicates, CI77007, pigment blue 29), chromium oxide hydrate (CI77289), iron blue (ferric ferrocyanide, CI77510), carmine (cochineal). Particular preference is given to pearlescent and color pigments based on mica which are coated with a metal oxide or a metal oxychloride such as titanium dioxide or bismuth oxychloride, and, if appropriate, further color-imparting substances such as iron oxides, iron blue, ultramarine, carmine etc. and where the color can be determined by varying the layer thickness. Such pigments are sold, for example, under the trade names Rona®, Colorona®, Dichrona® and Timiron® (Merck).

Organic pigments are, for example, the natural pigments sepia, gamboge, charcoal, Cassel brown, indigo, chlorophyll and other plant pigments. Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

In one embodiment, the composition according to the invention comprises 0.01 to 10% by weight, particularly preferably from 0.05 to 5% by weight, of at least one particulate substance. Suitable substances are, for example, substances which are solid at room temperature (25° C.) and are in the form of particles. For example, silica, silicates, aluminates, clay earths, mica, salts, in particular inorganic metal salts, metal oxides, e.g. titanium dioxide, minerals and polymer particles are suitable. The particles are present in the agent undissolved, preferably in stably dispersed form and, following application to the application surface and evaporation of the solvent, can settle out in solid form.

Preferred particulate substances are silica (silica gel, silicon dioxide) and metal salts, in particular inorganic metal salts, particular preference being given to silica. Metal salts are, for example, alkali metal or alkaline earth metal halides, such as sodium chloride or potassium chloride; alkali metal or alkaline earth metal sulfates, such as sodium sulfate or magnesium sulfate.

UV Filter Substances

In one preferred embodiment, the compositions according to the invention comprise oil-soluble and/or water-soluble UVA and/or UVB filters. Advantageously, the compositions comprise substances which absorb UV radiation in the UVB region and substances which absorb UV radiation in the UVA region, the total amount of filter substances being, for example, 0.1 to 30% by weight, preferably 0.5 to 20% by weight, in particular 1 to 15% by weight, based on the total weight of the compositions, in order to make available cosmetic compositions which protect hair, skin and scalp from the entire range of ultraviolet radiation.

The greatest part of the photoprotective agents in the cosmetic or dermatological compositions serving to protect the human epidermis consists of compounds which absorb UV light in the UV-B region. For example, the fraction of UV-A absorbers to be used according to the invention is 10 to 90% by weight, preferably 20 to 50% by weight, based on the total amount of UV-B and UV-A absorbing substances.

The UVB filters may be oil-soluble or water-soluble. Advantageous UVB filter substances are, for example:
benzimidazolesulfonic acid derivatives, such as, for example, 2-phenyl-benzimidazole-5-sulfonic acid and salts thereof
benzotriazole derivatives, such as, for example, 2,2′-methylenebis(6-(2H-benzotriazole-2-yl)-4-(1,1,3,3-tetramethylbutyl)phenol)
4-aminobenzoic acid derivatives, preferably 2-ethylhexyl 4-(dimethylamino)benzoate, amyl 4-(dimethylamino)benzoate;
esters of benzylmalonic acid, preferably di(2-ethylhexyl) 4-methoxybenzyl-malonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone;
methylidenecamphor derivatives, preferably 4-methylbenzylidenecamphor, benzylidenecamphor;
triazine derivatives, preferably tris(2-ethylhexyl) 4,4′,4″-(1,3,5-triazine-2,4,6-trianilino)trisbenzoate [INCl: Diethylhexyl Butamido Triazine, UVA-Sorb® HEB (Sigma 3V)] and 2,4,6-tris[anilino(p-carbo-2′-ethyl-1′-hexyloxy)]-1,3,5-triazine [INCl: Octyl Triazone, UVINUL®T 150 (BASF)].

Water-soluble UVB filter substances to be used advantageously are, for example, sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and salts thereof.

UVA filters to be used advantageously are, for example:
1,4-phenylenedimethinecamphorsulfonic acid derivatives, such as 3,3′-(1,4-phenylenedimethine)bis(7,7-dimethyl-2-oxobicyclo[2.2.1]heptane-1-methane-sulfonic acid and its salts
1,3,5-triazine derivatives, such as 2,4-bis{[(2-ethylhexyloxy)-2-hydroxy)-phenyl}-6-(4-methoxyphenyl)-1,3,5)-triazine (e.g. Tinosorb®S (Ciba))
dibenzoylmethane derivatives, preferably 4-isopropyldibenzoylmethane, 4-(tert-butyl)-4′-methoxydibenzoylmethane
benzoxazol derivatives, for example 2,4-bis[4-[5-(1,1-dimethyl-propyl)benzoxazol-2-yl]phenylimino]-6-[(2-ethylhexyl)imino]-1,3,5-triazine (CAS No. 288254-1 6-0, Uvasorb®K2A (3V Sigma))
hydroxybenzophenones, for example hexyl 2-(4′-diethylamino-2′-hydroxy-benzoyl)benzoate (also: aminobenzophenone) (Uvinul®A Plus (BASF))

In addition, according to the invention, it may, if appropriate, be advantageous to provide compositions with further UVA and/or UVB filters, for example certain salicylic acid derivatives, such as 4-isopropylbenzyl salicylate, 2-ethylhexyl salicylate, octyl salicylate, homomenthyl salicylate. The total amount of salicylic acid derivatives in the compositions according to the invention is advantageously chosen from the range from 0.1-15.0% by weight, preferably 0.3-10.0% by weight, based on the total weight of the compositions. A further photoprotective filter to be used advantageously according to the invention is ethylhexyl 2-cyano-3,3-diphenylacrylate (octocrylene, Uvinul®N 539 (BASF)).

The table below lists some photoprotective filters suitable for use in the compositions according to the invention:

For example, UV photoprotective filters to be mentioned are:

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one | 52793-97-2 |
| | methylsulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone (mexenone) | 1641-17-4 |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)- | 103597-45-1 |
| | 4-(1,1,3,3,-tetramethylbutyl)phenol] | |
| 30 | 2,2′-(1,4-Phenylene)bis-1H-benzimidazole-4,6-disulfonic acid, | 180898-37-7 |
| | Na salt | |
| 31 | 2,4-bis[4-(2-Ethylhexyloxy)-2-hydroxy]phenyl- | 187393-00-6 |
| | 6-(4-methoxyphenyl)(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone-5,5′-disodium | 3121-60-6 |
| | sulfonate | |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3- | 363602-15-7 |
| | butadiene | |

Polymeric or polymer-bound filter substances can also be used according to the invention.

Metal oxides, such as titanium dioxide or zinc oxide, are used widely in sunscreens. Their effect is essentially based on reflection, scattering and absorption of the harmful UV radiation and is essentially dependent on the primary particle size of the metal oxides. Furthermore, the according to the invention can comprise inorganic pigments based on metal oxides and/or other metal compounds which are insoluble or sparingly soluble in water, chosen from the group of oxides of zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. The pigments are particularly preferably based on ZnO.

The inorganic pigments can here be in coated form, i.e. be surface-treated. This surface treatment can, for example, consist in providing the pigments with a thin hydrophobic layer by a method known per se, as described in DE-A-33 14 742.

Photoprotective agents suitable for use in the compositions according to the invention are also the compounds specified in EP-A 1 084 696 in paragraphs [0036] to [0053], to which reference is made at this point in its entirety. Of suitability for the use according to the invention are all UV photoprotective filters which are specified in Annex 7 (at §3b) of the German Cosmetics Ordinance under “ultraviolet filters for cosmetic products”.

The list of specified UV photoprotective filters which can be used in the compositions according to the invention is not exhaustive.

Polymers

The compositions according to the invention can also comprise additional polymers. Suitable polymers are, for example, copolymers of vinylpyrrolidone/N-vinyl-imidazolium salts (Luviquat® FC, Luviquat® HM, Luviquat® MS, Luviquat® Care, Luviquat® UltraCare), copolymers of N-vinylpyrrolidone/dimethylaminoethyl methacrylate, quaternized with diethyl sulfate (Luviquat® PQ11), copolymers of N-vinylcaprolactam/N-vinylpyrrolidone/N-vinylimidazolium salts (Luviquat® Hold); cationic cellulose derivatives (Polyquaterhium-4, −10 and −67), acrylamido copolymers (Polyquaternium-7) and chitosan. Suitable cationic (quaternized) polymers are also Merquat® (polymer based on dimethyldiallylammonium chloride), Gafquat® (quaternary polymers which are formed by reacting polyvinylpyrrolidone with quaternary ammonium compounds), polymer JR (hydroxyethylcellulose with cationic groups) and cationic polymers based on plants, e.g. guar polymers, such as the Jaguar® grades from Rhodia.

Further suitable polymers are also neutral polymers, such as polyvinylpyrrolidones, copolymers of N-vinylpyrrolidone and vinyl acetate and/or vinyl propionate and/or stearyl(meth)acrylate, polysiloxanes, polyvinylcaprolactam and other copolymers with N-vinylpyrrolidone, copolymers with N-vinylformamide, and (partial) hydrolysates thereof, polyethyleneimines and salts thereof, polyvinylimines and salts thereof, cellulose derivatives, polysaccharide derivatives, polyaspartic acid salts and derivatives. These include, for example, Luviflex® Swing (partially hydrolyzed copolymer of polyvinyl acetate and polyethylene glycol, BASF) or Kollicoat® IR.

Suitable polymers are also the (meth)acrylamide copolymers described in WO 03/092640, in particular those described as examples 1 to 50 (table 1, page 40 ff.) and examples 51 to 65 (table 2, page 43), to which reference is hereby made in its entirety.

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 64 (BASF); polyamides, e.g. based on itaconic acid and aliphatic diamines, as described, for example, in DE-A-43 33 238.

Suitable polymers are also amphoteric or zwitterionic polymers, such as the octyl-acrylamide/methyl methacrylate/tert-butylaminoethyl methacrylate/2-hydroxypropyl methacrylate copolymers obtainable under the names Amphomer® (National Starch), and zwitterionic polymers as are disclosed, for example, in the German patent applications DE 39 29 973, DE 21 50 557, DE 28 17 369 and DE 37 08 451.

Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or-dispersible polymers, e.g. polyether siloxanes, such as Tegopren® (Goldschmidt) or Belsil® (Wacker).

Furthermore, biopolymers are also suitable, i.e. polymers which are obtained from naturally renewable raw materials and are constructed from natural monomer building blocks, e.g. cellulose derivatives, chitin, chitosan, DNA, hyaluronic acid and RNA derivatives.

Further preparations according to the invention comprise at least one further water-soluble polymer, in particular chitosans (poly(D-glucosamines)) of varying molecular weight and/or chitosan derivatives.

Anionic Polymers

Further polymers suitable for the preparations according to the invention are copolymers containing carboxylic acid groups. These are polyelectrolytes with a relatively large number of anionically dissociable groups in the main chain and/or one side chain.

Suitable polymers containing carboxylic acid groups are obtainable, for example, by free-radical polymerization of α,β-ethylenically unsaturated monomers. The process uses monomers m1) which comprise at least one free-radically polymerizable, α,β-ethylenically unsaturated double bond and at least one anionogenic and/or anionic group per molecule.

Suitable polymers containing carboxylic acid groups are also polyurethanes containing carboxylic acid groups. Preferably, the monomers are chosen from monoethylenically unsaturated carboxylic acids, sulfonic acids, phosphonic acids and mixtures thereof.

The monomers m1) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers also include the half-esters of monoethylenically unsaturated dicarboxylic acids having 4 to 10, preferably 4 to 6, carbon atoms, e.g. of maleic acid, such as monomethyl maleate. The monomers also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers also include the salts of the abovementioned acids, in particular the sodium, potassium and ammonium salts, and also the salts with the abovementioned amines. The monomers can be used as they are or as mixtures with one another. The stated weight fractions all refer to the acid form. Preferably, the monomer m1) is chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, particularly preferably acrylic acid, methacrylic acid and mixtures thereof.

The abovementioned monomers m1) can in each case be used individually or in the form of any mixtures.

Furthermore, the polymers containing carboxylic acid groups preferably comprise at least one monomer m2) in copolymerized form which is chosen from compounds of the general formula (VI)

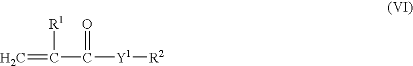

in which

R¹ is hydrogen or C₁-C₈-alkyl,

Y¹ is O, NH or NR³, and

R² and R³, independently of one another, are C₁-C₃₀-alkyl or C₅-C₈-cycloalkyl, where the alkyl groups may be interrupted by up to four nonadjacent heteroatoms or heteroatom-containing groups which are chosen from O, S and NH.

Preferably, R¹ in the formula VI is hydrogen or C₁-C₄-alkyl, in particular hydrogen, methyl or ethyl. Preferably, R² in the formula VI is C₁-C₈-alkyl, preferably methyl, ethyl, n-butyl, isobutyl, tert-butyl or a group of the formula —CH₂—CH₂—NH—C(CH₃)₃. If R³ is alkyl, then it is preferably C₁-C₄-alkyl, such as methyl, ethyl, n-propyl, n-butyl, isobutyl and tert-butyl.

Suitable monomers m2) are methyl(meth)acrylate, methyl ethacrylate, ethyl(meth)acrylate, ethyl ethacrylate, tert-butyl(meth)acrylate, tert-butyl ethacrylate, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arachinyl(meth)acrylate, behenyl(meth)acrylate, lignocerenyl(meth)acrylate, cerotinyl(meth)acrylate, melissinyl(meth)acrylate, palmitoyleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable monomers m2) are also acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide, N-(n-octyl)(meth)acrylamide, N-(1,1,3,3-tetramethyl-butyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide and N-lauryl(meth)acrylamide.

Furthermore, the polymers containing carboxylic acid groups preferably comprise at least one monomer m3) in copolymerized form which is chosen from compounds of the general formula VII

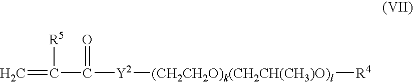

in which

the order of the alkylene oxide units is arbitrary,

k and l, independently of one another, are an integer from 0 to 1000, where the sum of k and l is at least 5,

R⁴ is hydrogen, C₁-C₃₀-alkyl or C₅-C₈-cycloalkyl,

R⁵ is hydrogen or C₁-C₈-alkyl,

Y² is O or NRS, where R⁶ is hydrogen, C₁-C₃₀-alkyl or C₅-C₈-cycloalkyl.

Preferably, in the formula VII, k is an integer from 1 to 500, in particular 3 to 250.

Preferably, l is an integer from 0 to 100. Preferably, R⁵ is hydrogen, methyl, ethyl, n-propyl, isopropyl, n-butyl, sec-butyl, tert-butyl, n-pentyl or n-hexyl, in particular hydrogen, methyl or ethyl. Preferably, R⁴ in the formula VII is hydrogen, methyl, ethyl, n-propyl, isopropyl, n-butyl, sec-butyl, n-pentyl, n-hexyl, octyl, 2-ethylhexyl, decyl, lauryl, palmityl or stearyl. Preferably, Y² in the formula VII is O or NH.

Suitable polyether acrylates VII) are, for example, the polycondensation products of the abovementioned α,β-ethylenically unsaturated mono- and/or dicarboxylic acids and their acid chlorides, amides and anhydrides with polyetherols. Suitable polyetherols can be readily prepared by reacting ethylene oxide, 1,2-propylene oxide and/or epichlorohydrin with a starter molecule, such as water or a short-chain alcohol R⁴—OH. The alkylene oxides can be used individually, alternately one after the other or as a mixture. The polyether acrylates VII) can be used on their own or in mixtures for preparing the polymers used according to the invention. Suitable polyether acrylates II) are also urethane (meth)acrylates with alkylene oxide groups. Such compounds are described in DE 198 38 851 (component e2)), to which reference is hereby made in its entirety.

Anionic polymers preferred as polymers containing carboxylic acid groups are, for example, homopolymers and copolymers of acrylic acid and methacrylic acid and salts thereof. These also include crosslinked polymers of acrylic acid, as are obtainable under the INCI name Carbomer. Such crosslinked homopolymers of acrylic acid are available commercially, for example, under the name Carbopol® from Noveon. Preference is also given to hydrophobically modified crosslinked polyacrylate polymers such as Carbopol® Ultrez 21 from Noveon.

Further examples of suitable anionic polymers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm and Haas. Particularly suitable polymers are also copolymers of t-butyl acrylate, ethyl acrylate, methacrylic acid (e.g. Luvimer®100P, Luvimer®Pro55), copolymers of ethyl acrylate and methacrylic acid (e.g. Luvimer®MAE), copolymers of N-tert-butylacrylamide, ethyl acrylate, acrylic acid (Ultrahold® 8, Ultrahold® Strong), copolymers of vinyl acetate, crotonic acid and, if appropriate, further vinyl esters (e.g. Luviset® grades), maleic anhydride copolymers, if appropriate reacted with alcohol, anionic polysiloxanes, e.g. carboxy-functional, t-butyl acrylate, methacrylic acid (e.g. Luviskol® VBM), copolymers of acrylic acid and methacrylic acid with hydrophobic monomers, such as, for example, C₄-C₃₀-alkyl esters of meth(acrylic acid), C₄-C₃₀-alkylvinyl esters, C₄-C₃₀-alkyl vinyl ethers and hyaluronic acid. Examples of anionic polymers are also vinyl acetate/crotonic acid copolymers, as are commercially available, for example, under the names Resyn® (National Starch) and Gafset® (GAF) and vinylpyrrolidone/vinyl acrylate copolymers obtainable, for example, under the trade name Luviflex® (BASF). Further suitable polymers are the vinylpyrrolidone/acrylate terpolymer available under the name Luviflex® VBM-35 (BASF) and sodium sulfonate-containing polyamides or sodium sulfonate-containing polyesters.

The group of suitable anionic polymers further comprises, by way of example, Balance® CR (National Starch; Acrylate Copolymer), Balance® 0/55 (National Starch; Acrylate Copolymer), Balance® 47 (National Starch; octylacrylamide/acrylate/butyl-aminoethyl methacrylate copolymer), Aquaflex® FX 64 (ISP; isobutylene/ethyl-maleimide/hydroxyethylmaleimide copolymer), Aquaflex® SF-40 (ISP/National Starch; VP/vinyl caprolactam (DMAPA acrylate copolymer), Allianz® LT-120 (ISP/Rohm & Haas; acrylate/C₁-2 succinate/hydroxyacrylate copolymer), Aquarez® HS (Eastman; polyester-1), Diaformer® Z-400 (Clariant; methacryloylethyl-betaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether/maleic acid in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC37 (ISP: terpolymer of vinylcaprolactam/vinylpyrrolidone/dimethylaminoethyl methacrylate), Advantage® LC55 and LC80 or LC A and LC E, Advantage® Plus (ISP; VA/butyl maleate/isobornyl acrylate copolymer), Aculyn® 258 (Rohm & Haas; acrylate/hydroxyl ester acrylate copolymer), Luviset® P.U.R. (BASF, polyurethane-1), Luviflex® Silk (BASF), Eastman® AQ 48 (Eastman), Styleze® CC-10 (ISP; VP/DMAPA acrylates copolymer), Styleze® 2000 (ISP; VP/acrylates/lauryl methacrylate copolymer), DynamX® (National Starch; polyurethane-14 AMP-acrylates copolymer), Resyn XP® (National Starch; acrylates/octylacrylamide copolymer), Fixomer® A-30 (Ondeo Nalco; polymethacrylic acid (and) acrylamidomethylpropanesulfonic acid), Fixate® G-100 (Noveon; AMP-acrylates/allyl methacrylate copolymer).

Suitable polymers containing carboxylic acid groups are also the terpolymers of vinylpyrrolidone/C₁-C₁₀-alkyl, cycloalkyl and aryl(meth)acrylates and acrylic acid described in U.S. Pat. No. 3,405,084. Suitable polymers containing carboxylic acid groups are also the terpolymers of vinylpyrrolidone, tert-butyl(meth)acrylate and (meth)acrylic acid described in EP-A-0 257 444 and EP-A-0 480 280. Suitable polymers containing carboxylic acid groups are also the copolymers described in DE-A-42 23 066 which comprise at least one (meth)acrylic ester, (meth)acrylic acid and N-vinylpyrrolidone and/or N-vinylcaprolactam in copolymerized form. Reference is hereby made to the disclosure of these documents in its entirety.

The abovementioned polymers containing carboxylic acid groups are prepared by known methods, for example solution, precipitation, suspension or emulsion polymerization.

Suitable polymers containing carboxylic acid groups are also polyurethanes containing carboxylic acid groups.

EP-A-636361 discloses suitable block copolymers with polysiloxane blocks and polyurethane/polyurea blocks which have carboxylic acid and/or sulfonic acid groups. Suitable silicone-containing polyurethanes are also described in WO 97/25021 and EP-A-751 162.

Suitable polyurethanes are also described in DE-A-42 25 045, to which reference is hereby made in its entirety.

The acid groups of the polymers containing carboxylic acid groups may be partly or completely neutralized. At least some of the acid groups are then in deprotonated form, the counterions preferably being chosen from alkali metal ions, such as Na⁺, K⁺, ammonium ions and organic derivatives thereof etc.

Shampoo Types

Depending on the hair quality or scalp problem, shampoos are, if appropriate, subject to additional requirements. The mode of action of the preferred shampoo types with the most important additional effects or most important specific objectives is described below.

According to the invention, preference is given, for example, to shampoos for normal or rapidly greasing or damaged hair, antidandruff shampoos, baby shampoos and two-in-one shampoos (shampoo and conditioner in one).

Shampoos for normal hair: hair washing should free hair and scalp from the skin sebum formed in sebaceous glans, the inorganic salts which emerge from sweat glands with water, amino acids, urea and lactic acid, skin particles which have flaked off, environmental dirt, odors, and, if appropriate, residues of hair cosmetic treatments. Normal hair means short to shoulder-length hair which is only slightly damaged. Accordingly, the fraction of conditioning auxiliaries should be optimized to this hair type.

Shampoos for rapidly greasing hair: increased sebum production of the sebaceous glands in the scalp leads even 1-2 days after hair washing to a straggly, unsightly hairstyle. Oil- and wax-like skin sebum constituents weigh down the hair and thus lower the friction from hair to hair and thus reduce the hairstyle hold. The actual hair cosmetic problem in the case of rapidly greasing hair is thus the premature collapse of voluminous hairstyles. In order to avoid this, it is necessary to prevent the surface of the hair becoming weighed down and too smooth and supple. This is preferably achieved through the surfactant base of washing raw materials which are good at cleaning and are marked by particularly low substantivity. Additional care substances which would add to the skin sebum, such as refatting substances or conditioning auxiliaries are only used in shampoos for rapidly greasing hair with the greatest of care, if at all. Voluminizing shampoos for fine hair can have a comparable formulation.

Shampoos for dry, stressed (damaged) hair. The structure of the hair is changed in the course of hair growth by mechanical influences such as combing, brushing and, in particular, back-combing (combing against the direction of growth), by the effect of UV radiation and visible light and by cosmetic treatments, such as permanent waves, bleaching or coloring. The scale layer of the hair has an increasingly stressed appearance from the root to the end; in extreme cases, it is completely worn away at the end, and the hair ends are split (split ends). In principle, it is no longer possible to return damaged hair to the state of healthy hair regrowth. However, it is possible to come very close to this ideal state with regard to feel, shine and combability by using shampoos according to the invention with, if appropriate, high fractions of care substances (conditioners).

An even better effect than with a shampoo is achieved with a haircare agent according to the invention, for example in the form of a rinse or cure treatment after hairwashing.

Two-in-one shampoos according to the invention in which, through the design as “shampoo and conditioner in one”, the additional care benefit is regarded as equivalent to the fundamental cleaning benefit. Two-in-one compositions according to the invention comprise increased amounts of the conditioners.

Antidandruff shampoos: compared to antidandruff hair tonics, antidandruff shampoos according to the invention have the advantage that they not only reduce the formation of new visible flakes as a result of appropriate active ingredients to combat an attack of dandruff and, in the case of long-term use, prevent such formation, but also remove scales which have already flaked off with hairwashing. After rinsing out the wash liquor, however, only a small, but sufficient amount of the active ingredients remains on scalp and hair. There are various antidandruff active ingredients which can be incorporated into the shampoo compositions according to the invention, such as, for example, zinc pyrithione, ketoconazole, clotrimazol, climbazol or pirocton olamine. In addition, these substances have a desquamation-normalizing effect. The basis of antidandruff shampoos corresponds largely to the formulation of shampoos for normal hair with a good cleaning effect.

Baby shampoos: in one preferred embodiment of the invention, the shampoo preparations according to the invention are baby shampoos. These are optimally skin- and mucosa-compatible. Combinations of washing raw materials with very good skin compatibility form the basis of these shampoos. Additional substances for further improving the skin and mucosa compatibility and the care properties are advantageously added, such as, for example, nonionic surfactants, protein hydrolysates and panthenol or bisabolol. All necessary raw materials and auxiliaries, such as preservatives, perfume oils, dyes etc., are chosen under the aspect of high compatibility and mildness.

Shampoos for dry scalp: in a further preferred embodiment of the invention, the shampoo preparations according to the invention are shampoos for dry scalp. The primary aim of these shampoos is to prevent the scalp from drying out since dry scalp can lead to irritation, reddening and inflammation. As in the case of the baby shampoos, combinations of washing raw materials with very good skin compatibility form the basis of these shampoos. Additionally, if appropriate, refatting agents and humectants, such as, for example, glycerol or urea, can be used.

The shampoo compositions according to the invention can also be in the form of shampoo concentrates with increased surfactant contents of 20-30%. They are based on specific washing raw material combinations and consistency regulators which ensure good dispersibility and spontaneous foaming ability even in a small amount. A particular advantage is, for example, the possibility of achieving the productivity of 200 ml of shampoo with a 100 ml bottle.

Presentation

It is advantageous if the cosmetic compositions according to the invention are stored in appropriate containers such as, for example, a bottle or squeezy bottle, and are used from these. Accordingly, bottles or squeezy bottles which comprise a composition according to the invention are also in accordance with the invention.

It is advantageous according to the invention if the cosmetic preparation according to the invention is stored in a bottle, squeezy bottle, pump spray or aerosol can, and used from this. Accordingly, bottles, squeezy bottles, pump spray or aerosol cans which comprise a preparation according to the invention are also in accordance with the invention.

The preparations according to the invention are advantageously used for caring for the hair, in particular the head hair.

Last but not least, the use of a cosmetic preparation according to the invention as hair shampoo and/or hair conditioner, i.e. as agent for conditioning the hair, is in accordance with the invention.

Skin Cosmetic Preparations

The polymers according to the invention can advantageously also be used in skin cosmetic preparations.

Particularly preferably, the compositions according to the invention are used in agents for cleaning the skin. Such cleaning agents are chosen from bar soaps, such as toilet soap, curd soaps, transparent soaps, luxury soaps, deodorant soaps, cream soaps, baby soaps, skin protection soaps, abrasive soaps and syndets, liquid soaps, such as pasty soaps, soft soaps and washing pastes, and liquid washing, showering and bath preparations, such as washing lotions, shower baths and gels, foam baths, oil baths and scrub preparations.

Shampoos and also washing, showering and bath preparations furthermore comprise surfactants. Preferred anionic, amphoteric and nonionic surfactants are specified, for example, in “Kosmetik and Hygiene von Kopf bis Fuβ” [Cosmetic and hygiene from head to toe“, Ed. W. Umbach, 3rd edition, Wiley-VCH, 2004, pp. 131-134, to which reference is made at this point in its entirety.

The compositions according to the invention are preferably used in cosmetic agents for the care and protection of the skin, in nailcare agents, and in preparations for decorative cosmetics.

The use in skincare agents, intimate care agents, footcare agents, deodorants, photoprotective agents, repellents, shaving agents, hair removers, antiacne agents, make-up, mascara, lipsticks, eyeshadows, kohl pencils, eyeliners, blushers, powders and eyebrow pencils is also preferred.

The skincare agents are present in particular as W/O or O/W skin creams, day and night creams, eye creams, face creams, antiwrinkle creams, humectant creams, bleach creams, vitamin creams, skin lotions, care lotions and moisturizing lotions.

Depending on the field of use, the agents according to the invention can be applied in a form suitable for skin care, such as, for example, as cream, foam, gel, stick, powder, mousse, milk or lotion. Further compositions according to the invention which may be mentioned are, for example, skin cosmetic preparations, in particular those for skin care. These are present in particular as W/O or O/W skin creams, day and night creams, eye creams, face creams, antiwrinkle creams, moisturizing creams, bleach creams, vitamin creams, skin lotions, care lotions and moisturizing lotions.

In addition, the compositions according to the invention are suitable for skin cosmetic preparations such as face tonics, face masks, deodorants and other cosmetic lotions and for use in decorative cosmetics, for example as concealing stick, stage make-up, in mascara and eyeshadows, lipsticks, kohl pencils, eyeliners, make-up, foundations, blushers and powders and eyebrow pencils.

Furthermore, the compositions according to the invention can be used in nose strips for pore cleansing, in antiacne agents, repellents, shaving agents, hair removers, intimate care agents, footcare agents and in babycare.

Besides the polymers a) and b) and suitable carriers, the skin cosmetic preparations according to the invention can also comprise further active ingredients and auxiliaries customary in skin cosmetics, as described above for hair cosmetic preparations and below.

These include preferably emulsifiers, preservatives, perfume oils, cosmetic active ingredients such as phytantriol, vitamin A, E and C, retinol, bisabolol, panthenol, natural and synthetic photoprotective agents, bleaches, colorants, tints, tanning agents, collagen, protein hydrolysates, stabilizers, pH regulators, dyes, salts, thickeners, gel formers, consistency regulators, silicones, humectants, conditioners, refatting agents and further customary additives.

Further conventional polymers may also be added to the compositions according to the invention if specific properties are to be set. To set certain properties, such as, for example, changing the feel to the touch, the spreading behavior, the water resistance and/or the binding of active ingredients and auxiliaries such as pigments, the compositions can additionally also comprise further conditioning substances based on silicone compounds. Suitable silicone compounds are, for example, polyalkylsiloxanes, polyarylsiloxanes, polyarylalkylsiloxanes, polyether siloxanes or silicone resins.

In one embodiment of the invention, the compositions according to the invention, however, comprise no further conditioning polymers.

The compositions according to the invention comprising polymer a) and polymer b) are suitable for enhancing or increasing the deposition amount and rate and also the residence time of further active ingredients likewise present, if appropriate, in these compositions according to the invention, such as, for example, silicones or UV photoprotective filters, on the skin and/or the hair. Substances or agents which have such effects are also referred to as depositioning aids.

U.S. Pat. No. 6,998,113 describes rinse-off preparations which lead to the skin treated therewith being effectively protected against UV radiation. Some of the preparations described therein comprise cationic polymers. For the purposes of the present invention, the mixtures according to the invention comprising the at least one cationic polymer a) and the at least one water-soluble polymer b) can also be used in the preparations of U.S. Pat. No. 6,998,113. In particular, the mixtures according to the invention can be used for the purpose specified by U.S. Pat. No. 6,998,113 in sunscreen, washing and bathing preparations. Reference is hereby made to the disclosure of U.S. Pat. No. 6,998,113 in its entirety.

Suitable silicones are described, for example, in U.S. Pat. No. 5,935,561, column 13, l. 64 to column 18, l. 61, to which reference is hereby made in its entirety. By way of representation, mention may be made of:
dimethicones
polyalkyl- or polyarylsiloxanes (U.S. Pat. No. 5,935,561), column 13, formula I)
alkylamino substituted silicones (U.S. Pat. No. 5,935,561, column 14, formula II (amodimethicone))
cationic silicones (U.S. Pat. No. 5,935,561, columns 14 and 15, formula III)
trimethylsilylamodimethicones (U.S. Pat. No. 5,935,561, column 15, formula IV)
silicones according to U.S. Pat. No. 5,935,561, column 15, formula V
cyclic polysiloxanes according to U.S. Pat. No. 5,935,561, column 16, formula VI

Measurement Methods

Determination of the K Values

The K values were measured in accordance with Fikentscher, Celluloseschemie [Cellulose chemistry], Vol. 13, p. 58 to 64 (1932) at 25° C. in aqueous solution and are a measure of the molecular weight. The measurement solutions of the polymers each comprise 1 g of the respective polymer in 100 ml of solution, the solvent used being completely demineralized water (dem. water). The K values were measured in a micro-Ubbelohde capillary type M Ic from Schott.

All of the samples were treated as follows:
The polymer mixture according to the invention in each case was dissolved in an amount of about 0.5% by weight in water. The remaining ingredients were then added and a homogeneous mixture was produced.
To establish any phase separation (stability test), the mixtures produced in this way were subjected to a centrifuge test;
the mixtures were then stored for up to 3 months at 40° C. and analyzed at regular intervals by inspection and centrifuge test for their stability with regard to phase separation.

Wet Combability

Determination of the Wet Combability (European Bleached Hair Tress)

Formulation of the shampoos based on the polymer mixtures according to the invention

| | | |
|---|---|---|
| 35.70 | g | Texapon ®NSO |
| 12.50 | g | Tego ®Betain L 7 |
| 1 | g | 20% by weight aqueous solution of the polymer mixture |
| 0.10 | g | Euxyl ®K 100 |
| 1.00 | g | NaCl |
| ad 100 | g | Water |

Wet Combability Blank Determination

Before the determination, a hair tress (length about 24 cm/weight 2.7-3.3 g) was firstly washed twice within a minute using Texapon®NSO and rinsed for 1 minute and squeezed on filter paper in order to achieve a defined wetness and swelling of the. The tress was then precombed so that knots were no longer present in the hair. The tress was then fixed to the holder and combed into the finely toothed side of the test comb using the finely toothed side of the comb. Insertion of the hair into the test comb was carried out evenly and without tension for each measurement. The measurement was started and evaluated using the EGRANUDO® software (Frank). The measurement was repeated 5-10 times. The measurements were carried out in a climatically controlled room at a relative humidity of about 65% and 21° C. The calculated average was noted together with the standard deviation.

Wet Combability Measurement Determination:

After determining the blank, about 5 g of the shampoo to be tested were applied to the hair tress to be tested, shampooed for 1 minute, rinsed for 1 minute, squeezed on filter paper and combed. The combing force was measured analogously to the blank determination described above.

Evaluation: Decrease in combing force wet=100−(measurement*100/blank); data in %

Instruments used:

Tensile/pressure testing instrument from Frank

Digital balance (top-pan balance)

Feel

During the shampooing operation of 1 minute (see above “wet combability measurement determination”), the foaming behavior, the foam creaminess, the care behavior and the foam volume is assessed. Afterwards, the tress is rinsed for 1 minute under running drinking water (shower spray). Using one hand, the hair tress is held open at the seam under the shower spray, with the other hand the hair is allowed to slide between thumb and palm from top to bottom. By wearing rubber gloves it is possible to sense how the hair feels as it slides along.

The waxy feel (silicone feel) of the wet hair is assessed subjectively
no waxy feel, (like untreated hair) inhibitory (+) corresponds to “poor”
slightly waxy, easy sliding (++) corresponds to “good”
waxy, very easy sliding (+++) corresponds to “very good”

The rinsed hair tress is squeezed between middle finger and forefinger, pressed on filter paper, combed and clamped into the apparatus.

Differences are detected upon combing into the combing device, i.e. the waxier the feel on the hair, the lower the resistance upon combing into the test comb. The combing force is measured analogously to the blank determination.

### EXAMPLES

The mixtures described in table 3 below were prepared by mixing aqueous solutions of the respective polymers corresponding to the desired quantitative weight ratios with stirring. After mixing, water was added until a solids content of the solution of about 30% by weight was reached. The mixture obtained was then stirred for a further hour.

TABLE 3
Examples of compositions according to the invention
Weight ratios are based on solids.

| | Composition | Weight | | | | | |
|---|---|---|---|---|---|---|---|
| | 0.2% by weight of the polymer | ratio | Wet | | Storage | Storage | Storage |
| Example | mixture in shampoo | a) to b) | compatibility | Feel | stability 1 | stability 2 | stability 3 |
| 001 | a) Poly-DADMAC^{#} and | 50:50 | 78% | good | yes | yes | yes |
| | b) Poly-VI | | | | | | |
| 002 | a) Poly-DADMAC^{#} and | 25:75 | 53% | good | yes | yes | no |
| | b) VP-VI-(50:50) K value 17 | | | | | | |
| 003 | a) Poly-DADMAC^{#} and | 75:25 | 56% | good | yes | no | no |
| | b) VP-VI-(50:50) K value 17 | | | | | | |
| 004 | a) Poly-DADMAC^{#} and | 90:10 | 61% | good | yes | no | no |
| | b) VP-VI-(50:50) K value 17 | | | | | | |
| 005 | a) Poly-DADMAC^{##} and | 55:45 | 78% | good | yes | yes | yes |
| | b) VP-VI-(50:50) K value 34 | | | | | | |
| 006 | a) Poly-DADMAC^{#} and | 50:50 | 71% | good | yes | yes | yes |
| | b) VI-VP-PEG; K value 38 | | | | | | |
| 007 | a) Poly-DADMAC^{#} and | 75:25 | 76% | good | yes | no | no |
| | b) VI-VP-PEG; K value 38 | | | | | | |
| 008 | a) Poly-DADMAC^{#} and | 90:10 | 83% | good | yes | no | no |
| | b) VI-VP-PEG; K value 38 | | | | | | |
| 009 | a) Poly-DADMAC^{#} and | 50:50 | 65% | good | yes | yes | no |
| | b) VP-VI-(50:50) K value 32 | | | | | | |
| 0010 | a) Poly-DADMAC^{#} and | 75:25 | 68% | good | yes | yes | no |
| | b) VP-VI-(50:50) K value 32 | | | | | | |
| 011 | a) Poly-DADMAC^{#} and | 90:10 | 82% | good | yes | no | no |
| | b) VP-VI (50:50) K value 32 | | | | | | |
| C01 | Poly-DADMAC^{#} | | 80% | good | no | no | no |
| | (02% by wt. in the shampoo) | | | | | | |
| C02 | Poly-DADMAC^{#} | | 50% | good | yes | no | no |
| | (01% by wt. in the shampoo) | | | | | | |
| C03 | PVI | | 30% | bad | yes | yes | no |
| | (0.2% by wt. in the shampoo) | | | | | | |
| C04 | PVI | | 30% | bad | yes | yes | no |
| | (0.1% by wt. in the shampoo) | | | | | | |

Explanation of terms for table 3:
Storage stability 1: Shampoos are stable after storage for 2 weeks at 40° C.
Storage stability 2: Shampoos are stable after storage for 3 months at 40° C.
Storage stability 3: Shampoos are stable after storage for 6 months at 40° C.

where “stable” means: optical homogeneity of the formulation, i.e. neither visible multiphase character nor particles.

#Poly-DADMAC: 30% by weight aqueous polyDADMAC solution with K value 90 (K value measured as 1% strength by weight aqueous solution)

##Poly-DADMAC: 30% by weight aqueous polyDADMAC solution with K value 81 (K value measured as 1% strength by weight aqueous solution)

Poly-VI: Polyvinylimidazole from example 1: polyvinylimidazole with K value 27 (K value measured as 1% strength by weight aqueous solution)

generally:

VP-VI-(x:y): Polymer of vinylpyrrolidone (VP) and vinylimidazole (VI) in the weight ratio x:y; VP-VI-(50:50) accordingly means that 50 parts by weight of VP and 50 parts by weight of VI are present in the polymer in incorporated form.

VP-VI-(50:50) K value 17: Polymer of vinylpyrrolidone and vinylimidazole in the weight ratio 1:1 with K value 17 (K value measured as 1% strength by weight aqueous solution)

VI-VP-PEG K value 38: Copolymer of vinylpyrrolidone and vinylimidazole copolymerized in the presence of polyethylene glycol, where the polymer has a K value of 38 (K value measured as 1% strength by weight aqueous solution)

VP-VI-(50:50) K value 32: Polymer of vinylpyrrolidone and vinylimidazole in the weight ratio 1:1 with K value 32 (K value measured as 1% strength by weight aqueous solution).

VP-VI-(50:50) K value 34: Polymer of vinylpyrrolidone and vinylimidazole in the weight ratio 1:1 with K value 34 (K value measured as 1% strength by weight aqueous solution).

Transmission

Conditions:

Instrument: Dr. Lange colorimeter, model Lico 200

Wavelength: 650 nm

Cells: 10 mm

Standard: water 100% transmission

All solutions at 10% to 40% Polymer content: Transmission>98%

Examples of Cosmetic Compositions:

Hair Cosmetic Composition (General)

a) 0.01 to 5% by weight of a mixture according to the invention

b) 25 to 99.99% by weight of water and/or alcohol

c) 0 to 95.99% by weight of further constituents

Alcohol is to be understood as meaning all alcohols customary in cosmetics, e.g. ethanol, isopropanol, n-propanol.

Further constituents are to be understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances such as panthenol, collagen, vitamins, protein hydrolysates, α- and β-hydroxycarboxylic acids, chitosan, protein hydrolysates, cosmetic polymers, stabilizers, pH regulators, dyes, viscosity regulators, gel formers, dyes, salts, humectants, refatting agents, complexing agents and further customary additives.

Shampoo Formulation/Shower Gel Formulation

Preferred shampoo formulations or shower gel formulations comprise

a) 0.01 to 5% by weight of a mixture according to the invention

b) 25 to 99.99% by weight of water

c) 0-5% by weight of a further conditioner

d) 0-30% by weight of further cosmetic constituents

In addition, in the shampoo formulations it is possible to use all anionic, neutral, amphoteric or cationic surfactants used customarily in shampoos with the above provisos.

### Example 1

### Conditioner Shampoo with PQ-10

| | | |
|---|---|---|
| 35.70 | g | Sodium Laureth Sulfate |
| 6.50 | g | Cocamidopropyl Betaine |
| 0.20 | g | of the mixture as in table 3, example 1 |
| 0.40 | g | Polyquaternium-10 |
| 0.10 | g | Preservative |
| 0.10 | g | Perfume oil/essential oil |
| ad 100 | g | Aqua dem. |

Good conditioner shampoos are also obtained if, instead of the mixture as in table 3, example 1, one or more of the mixtures as in table 3, examples 2 to 11 are used.

### Example 2

### Conditioner Shampoo with GHTC

| | | |
|---|---|---|
| 35.70 | g | Sodium Laureth Sulfate |
| 6.50 | g | Cocamidopropyl Betaine |
| 0.50 | g | of the mixture as in table 3, example 1 |
| 0.20 | g | Guarhydroxypropyltrimonium Chloride |
| 0.10 | g | Preservative |
| 0.10 | g | Perfume oil/essential oil |
| ad 100 | g | Aqua dem. |

Good conditioner shampoos are also obtained if, instead of the mixture as in table 3, example 1, one or more of the mixtures as in table 3, examples 2 to 11 are used.

### Example 3

### Conditioner Shampoo with Polyquaternium

| | | |
|---|---|---|
| 35.70 | g | Sodium Laureth Sulfate |
| 6.50 | g | Cocamidopropyl Betaine |
| 0.20 | g | of the mixture as in table 3, example 1 |
| 0.30 | g | Polyquaternium-44 or polyquaternium-67 |
| 0.10 | g | Preservative |
| 0.10 | g | Perfume oil/essential oil |
| ad 100 | g | Aqua dem. |

Good conditioner shampoos are also obtained if, instead of the mixture as in table 3, example 1, one or more of the mixtures as in table 3, examples 2 to 11 are used.

### Example 4

### Shampoo

Phase A

| | |
|---|---|
| 15.00 g | Cocamidopropyl Betaine |
| 10.00 g | Disodium Cocoamphodiacetate |
| 5.00 g | Polysorbate 20 |
| 5.00 g | Decyl Glucoside |
| 0.20 g | of the mixture as in table 3, example 1 |
| 0.10 g | Perfume oil/essential oil |
| q.s. | Preservative |
| 2.00 g | Laureth-3 |
| ad 100 | Aqua dem. |
| q.s. | Citric Acid |

Phase B

| | |
|---|---|
| 3.00 g | PEG-150 Distearate |

Preparation

Weigh in components of phase A and dissolve; adjust pH to 6-7. Add phase B and heat to 50° C. Allow to cool to room temperature with stirring.

Good shampoos are also obtained if, instead of the mixture as in table 3, example 1, one or more of the mixtures as in table 3, examples 2 to 11 are used.

### Example 5

### Shampoo

| | |
|---|---|
| 30.00 g | Sodium Laureth Sulfate |
| 6.00 g | Sodium Cocoamphoacetate |
| 0.50 g | of the mixture as in table 3, example 1 |
| 3.00 g | Sodium Laureth Sulfate, Glycol Distearate, |
| | Cocamide MEA, Laureth-10 |
| 2.00 g | Dimethicone |
| q.s. | Perfume |
| q.s. | Preservative |
| q.s. | Citric Acid |
| 1.00 g | Sodium Chloride |
| ad 100 | Aqua dem. |

Good shampoos are also obtained if, instead of the mixture as in table 3, example 1, one or more of the mixtures as in table 3, examples 2 to 11 are used.

### Example 6

### Shower Gel

| | |
|---|---|
| 20.00 g | Ammonium Laureth Sulfate |
| 15.00 g | Ammonium Lauryl Sulfate |
| 0.50 g | of the mixture as in table 3, example 1 |
| 0.50 g | Polyquaternium-7 |
| 2.50 g | Sodium Laureth Sulfate, Glycol Distearate, |
| | Cocamide MEA, Laureth-10 |
| 0.10 g | Perfume oil/essential oil |
| q.s. | Preservative |
| 0.50 g | Sodium Chloride |
| ad 100 | Aqua dem. |

Good shower gels are also obtained if, instead of the mixture as in table 3, example 1, one or more of the mixtures as in table 3, examples 2 to 11 are used.

### Example 7

### Shower Gel

| | |
|---|---|
| 40.00 g | Sodium Laureth Sulfate |
| 5.00 g | Decyl Glucoside |
| 5.00 g | of the mixture as in table 3, example 1 |
| 1.00 g | Panthenol |
| 0.10 g | Perfume oil/essential oil |
| q.s. | Preservative |
| q.s. | Citric Acid |
| 2.00 g | Sodium Chloride |
| ad 100 | Aqua dem. |

Good shower gels are also obtained if, instead of the mixture as in table 3, example 1, one or more of the mixtures as in table 3, examples 2 to 11 are used.

### Example 8

### Shampoo

| | |
|---|---|
| 12.00 g | Sodium Laureth Sulfate |
| 1.50 g | Decyl Glucoside |
| 0.50 g | of the mixture as in table 3, example 1 |
| 5.00 g | Coco-Glucoside Glyceryl Oleate |
| 2.00 g | Sodium Laureth Sulfate, Glycol Distearate, |
| | Cocamide MEA, Laureth-10 |
| q.s. | Preservative |
| q.s. | Sunset Yellow C.I. 15 985 |
| 0.10 g | Perfume oil/essential oil |
| 1.00 g | Sodium Chloride |
| ad 100 | Aqua dem. |

Good shampoos are also obtained if, instead of the mixture as in table 3, example 1, one or more of the mixtures as in table 3, examples 2 to 11 are used.

The mixtures according to the invention are also suitable in hairstyling preparations, in particular hair foams (aerosol foams with propellant gas and pump foams without propellant gas), hairsprays (pump sprays without propellant gas) and hair gels.

Propellants are the customarily used propellants. Preference is given to mixtures of propane/butane, pentane, dimethyl ether, 1,1-difluoroethane (HFC-152 a), carbon dioxide, nitrogen or compressed air.

Aerosol Hair Foam

a) 0.1 to 10% by weight of a cosmetic polymer

b) 55 to 99.8% by weight of water and/or alcohol

c) 5 to 20% by weight of a propellant

d) 0.1 to 5% by weight of the mixture according to the invention

e) 0 to 10% by weight of further constituents

Further constituents which may be used are, inter alia, all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl ester of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, quaternium-1 to x (INCI).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, α-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels may, for example, have the following composition:

### Example 9

### Aerosol Hair Foam

| | |
|---|---|
| 2.00 g | Cocotrimonium Methosulfate |
| 0.10 g | Perfume oil/essential oil |
| 3.50 g | Setting polymer e.g. polyquaternium-46, PQ-44, VP/ |
| | methacrylamide/vinylimidazole copolymer, etc. |
| 0.80 g | of the mixture as in table 3, example 1 |
| q.s. | Preservative |
| 75.00 g | Water dem. |
| 10.00 g | Propane/Butane (3.5 bar) |

Good aerosol hair foams are also obtained if, instead of the mixture as in table 3, example 1, one or more of the mixtures as in table 3, examples 2 to 11 are used.

Styling Gel

a) 0.1 to 10% by weight of a cosmetic polymer

b) 60 to 99.85% by weight of water and/or alcohol

c) 0.05 to 10% by weight of a gel former

d) 0.1 to 5% by weight of the mixture according to the invention

e) 0 to 20% by weight of further constituents

Gel formers which may be used are all gel formers customary in cosmetics. These include slightly crosslinked polyacrylic acid, for example Carbomer (INCI), cellulose derivatives, e.g. ethoxypropylcellulose, hydroxyethylcellulose, cationically modified celluloses, polysaccharides, e.g. xanthan gum, caprylic/capric triglyceride, sodium acrylate copolymer, polyquaternium-32 (and) Paraffinum Liquidum (INCI), Sodium Acrylates Copolymer (and) Paraffinum Liquidum (and) PPG-1 Trideceth-6, Acrylamidopropyl Trimonium Chloride/Acrylamide Copolymer, Steareth-10 Allyl Ether Acrylates Copolymer, Polyquatemium-37 (and) Paraffinum Liquidum (and) PPG-1 Trideceth-6, Polyquaternium-37 (and) Propylene Glycols Dicaprate Dicaprylate (and) PPG-1 Trideceth-6, Polyquaternium-7, Polyquaternium-44, Polyquaternium-67.

Good styling gels are also obtained if, instead of the mixture as in table 3, example 1, one or more of the mixtures as in table 3, examples 2 to 11 are used.

### Example 10

### Hairstyling Gel

Phase A

| | |
|---|---|
| 0.50 g | Carbomer or Acrylates/C10-30 Alkyl Acrylate Crosspolymer |
| 86.40 g | Water dem. |

Phase B

| | |
|---|---|
| 0.70 g | Triethanolamine |

Phase C

| | |
|---|---|
| 6.00 g | Setting polymer e.g. VP/Methacrylamide/ |
| | Vinyl Imidazole Copolymer |
| 5.00 g | PVP |
| 0.20 g | PEG-25 PABA |
| 0.50 g | of the mixture as in table 3, example 1 |
| 0.10 g | Perfume oil/essential oil |
| q.s. | PEG-14 Dimethicone |
| q.s. | Preservative |
| 0.10 g | Tocopheryl Acetate |

Good styling gels are also obtained if, instead of the mixture as in table 3, example 1, one or more of the mixtures as in table 3, examples 2 to 11 are used.

### Example 11

### Hairstyling Gel

Phase A

| | |
|---|---|
| 0.50 g | Carbomer or Acrylates/C10-30 Alkyl Acrylate Crosspolymer |
| 91.20 g | Water dem. |

Phase B

| | |
|---|---|
| 0.90 g | Tetrahydroxypropyl Ethylenediamine |

Phase C

| | |
|---|---|
| 7.00 g | VP/VA copolymer |
| 0.40 g | of the mixture as in table 3, example 1 |
| 0.20 g | Perfume oil/essential oil |
| q.s. | Preservative |
| 0.10 g | Propylene Glycol |

Good styling gels are also obtained if, instead of the mixture as in table 3, example 1, one or more of the mixtures as in table 3, examples 2 to 11 are used.

### Example 12

### Hair Wax Cream

| 6.00 | g | Caprylic/Capric Triglyceride |
|---|---|---|
| 3.00 | g | Glyceryl Stearate |
| 2.00 | g | Cetyl Alcohol |
| 3.50 | g | of the mixture as in table 3, example 1 |
| 0.50 | g | Cremophor A6 |
| 0.70 | g | Cremophor A25 |
| 0.50 | g | Dimethicone |
| 0.50 | g | Vitamin E Acetate |
| 2.00 | g | Caprylic/Capric Triglyceride and |
| | | Sodium Acrylates Copolymer |
| 1.00 | g | D-Panthenol USP |
| 0.10 | g | EDTA |
| 10.00 | g | Setting polymer |
| q.s. | | Preservative |
| ad 100 | g | Water dem. |

Good hair wax creams are also obtained if, instead of the mixture as in table 3, example 1, one or more of the mixtures as in table 3, examples 2 to 11 are used.

### Example 13

### Hair Pudding

| 3.00 | g | Kollicoat IR (BASF) |
|---|---|---|
| q.s. | | Preservative |
| 2.00 | g | Setting polymer |
| 4.00 | g | Acrylates/Beheneth-25 Methacrylate Copolymer |
| 0.70 | g | of the mixture as in table 3, example 1 |
| 0.50 | g | Dimethicone Copolyol |
| 0.10 | g | EDTA |
| 0.20 | g | Benzophenone-4 |
| ad 100 | g | Water dem. |

Good hair puddings are also obtained if, instead of the mixture as in table 3, example 1, one or more of the mixtures as in table 3, examples 2 to 11 are used.

### Example 14

### Spray Gel

Phase A

| | |
|---|---|
| 1.25 g | Setting polymer |
| 96.15 g | Aqua dem. |

Phase B

| | |
|---|---|
| 0.70 g | Acrylates/Steareth-20 Itaconate Copolymer |
| 0.10 g | Propylene Glycol |
| 0.50 g | of the mixture as in table 3, example 1 |
| 0.10 g | Glycerol |
| 0.10 g | Perfume oil/essential oil |
| q.s. | Preservative |

Phase C

| | |
|---|---|
| 0.70 g | Triethanolamine |

Good spray gels are also obtained if, instead of the mixture as in table 3, example 1, one or more of the mixtures as in table 3, examples 2 to 11 are used.

A preparation suitable according to the invention for styling sprays can, for example, have the following composition:

### Example 15

### Pump Hairspray

| | |
|---|---|
| 11.20 g | PEG/PPG-25/25 Dimethicone/Acrylates Copolymer |
| 2.80 g | VP/VA Copolymer |
| 1.34 g | Aminomethyl Propanol |
| 0.30 g | of the mixture as in table 3, example 1 |
| 0.10 g | Perfume oil/essential oil |
| 11.26 g | Aqua dem. |
| 73.00 g | Alcohol |

Good pump hairsprays are also obtained if, instead of the mixture as in table 3, example 1, one or more of the mixtures as in table 3, examples 2 to 11 are used.

### Example 16

### Pump Hairspray VOC55

| | |
|---|---|
| 2.00 g | VP/Methacrylamide/Vinyl Imidazole Copolymer |
| 1.90 g | Polyquaternium-46 |
| 2.00 g | of the mixture as in table 3, example 1 |
| 0.10 g | Perfume oil/essential oil |
| 55.00 g | Alcohol |
| 39.00 g | Aqua dem. |

Good pump hairsprays VOC 55 are also obtained if, instead of the mixture as in table 3, example 1, one or more of the mixtures as in table 3, examples 2 to 11 are used.

Skin Cosmetic Compositions

### Example 17

### Liquid Makeup

Phase A

| | |
|---|---|
| 1.70 g | Glyceryl Stearate |
| 1.70 g | Cetyl Alcohol |
| 1.70 g | Ceteareth-6 |
| 1.70 g | Ceteareth-25 |
| 5.20 g | Caprylic/Capric Triglyceride |
| 5.20 g | Mineral Oil or Luvitol ® Lite |
| | (INCI Hydrogenated Polyisobutene) |

Phase B

| | |
|---|---|
| q.s. | Preservative |
| 4.30 g | Propylene Glycol |
| 2.50 g | of the mixture as in table 3, example 1 |
| 59.50 g | Aqua dem. |

Phase C

| | |
|---|---|
| 0.10 g | Perfume oil/essential oil |

Phase D

| | |
|---|---|
| 2.00 g | Iron Oxides |
| 12.00 g | Titanium Dioxide |

Good liquid makeups are also obtained if, instead of the mixture as in table 3, example 1, one or more of the mixtures as in table 3, examples 2 to 11 are used.

### Example 18

### Eyeliner

Phase A

| | |
|---|---|
| 40.60 g | dist. water |
| 0.20 g | Disodium EDTA |
| q.s. | Preservative |

Phase B

| | |
|---|---|
| 0.60 g | Xanthan Gum |
| 0.40 g | Veegum |
| 3.00 g | Butylene Glycol |
| 0.20 g | Polysorbate-20 |

Phase C

| | |
|---|---|
| 15.00 g | Iron oxide/Al Powder/Silica (e.g. |
| | Sicopearl ® Fantastico Gold from BASF) |

Phase D

| | |
|---|---|
| 10.00 g | Aqua dem. |
| 25.00 g | Setting polymer (e.g. Polyurethane-1 or VP/ |
| | Methacrylamide/Vinyl Imidazole Copolymer, etc.) |
| 5.00 g | of the mixture as in table 3, example 1 |

Good eyeliners are also obtained if, instead of the mixture as in table 3, example 1, one or more of the mixtures as in table 3, examples 2 to 11 are used.

### Example 19

### Sunscreen Gel

Phase A

| | |
|---|---|
| 0.90 g | of the mixture as in table 3, example 1 |
| 8.00 g | Octyl Methoxycinnamate |
| 5.00 g | Octocrylene |
| 0.80 g | Octyl Triazone |
| 2.00 g | Butyl Methoxydibenzoylmethane |
| 2.00 g | Tocopheryl Acetate |
| 0.10 g | Perfume oil/essential oil |

Phase B

| | |
|---|---|
| 0.30 g | Acrylates/C10-30 Alkyl Acrylate Crosspolymer |
| 0.20 g | Carbomer |
| 5.00 g | Glycerol |
| 0.20 g | Disodium EDTA |
| q.s. | Preservative |
| 75.30 g | Aqua dem. |

Phase C

| | |
|---|---|
| 0.20 g | Sodium Hydroxide |

Good sunscreen gels are also obtained if, instead of the mixture as in table 3, example 1, one or more of the mixtures as in table 3, examples 2 to 11 are used.

### Example 20

### Sunscreen Emulsion with TiO₂ and ZnO₂

Phase A

| | |
|---|---|
| 1.00 g | PEG-7 Hydrogenated Castor Oil |
| 5.00 g | of the mixture as in table 3, example 1 |
| 2.00 g | PEG-45/Dodecyl Glycol Copolymer |
| 3.00 g | Isopropyl Myristate |
| 7.90 g | Jojoba (Buxus Chinensis) Oil |
| 4.00 g | Octyl Methoxycinnamate |
| 2.00 g | 4-Methylbenzylidene Camphor |
| 3.00 g | Titanium Dioxide, Dimethicone |
| 1.00 g | Dimethicone |
| 5.00 g | Zinc Oxide, Dimethicone |

Phase B

| | |
|---|---|
| 0.20 g | Disodium EDTA |
| 5.00 g | Glycerol |
| q.s. | Preservative |
| 60.80 g | Aqua dem. |

Phase C

| | |
|---|---|
| 0.10 g | Perfume oil/essential oil |

Good sunscreen emulsions are also obtained if, instead of the mixture as in table 3, example 1, one or more of the mixtures as in table 3, examples 2 to 11 are used.

### Example 21

### Face Tonic

Phase A

| | |
|---|---|
| 3.00 g | of the mixture as in table 3, example 1 |
| 0.10 g | Perfume oil/essential oil |
| 0.30 g | Bisabolol |

Phase B

| | |
|---|---|
| 3.00 g | Glycerol |
| 1.00 g | Hydroxyethyl Cetyldimonium Phosphate |
| 5.00 g | Whitch Hazel (Hamamelis Virginiana) Distillate |
| 0.50 g | Panthenol |
| q.s. | Preservative |
| 87.60 g | Aqua dem. |

Good face tonics are also obtained if, instead of the mixture as in table 3, example 1, one or more of the mixtures as in table 3, examples 2 to 11 are used.

### Example 22

### Face Washing Paste with Peeling Effect

Phase A

| | |
|---|---|
| 73.00 g | Aqua dem. |
| 1.50 g | Carbomer |
| q.s. | Preservative |

Phase B

| | |
|---|---|
| q.s. | Perfume oil |
| 7.00 g | Potassium Cocoyl Hydrolyzed Protein |
| 4.00 g | of the mixture as in table 3, example 1 |

Phase C

| | |
|---|---|
| 1.50 g | Triethanolamine |

Phase D

| | |
|---|---|
| 13.00 g | Polyethylene (Luwax A ™ from BASF) |

Good face washing pastes are also obtained if, instead of the mixture as in table 3, example 1, one or more of the mixtures as in table 3, examples 2 to 11 are used.

### Example 23

### Soap

Phase A

| | |
|---|---|
| 25.00 g | Potassium Cocoate |
| 20.00 g | Disodium Cocoamphodiacetate |
| 2.00 g | Lauramide DEA |
| 1.00 g | Glycol Stearate |
| 2.00 g | of the mixture as in table 3, example 1 |
| 50.00 g | Aqua dem. |
| q.s. | Citric Acid |

Phase B

| | |
|---|---|
| q.s. | Preservative |
| 0.10 g | Perfume oil/essential oil |

Good soaps are also obtained if, instead of the mixture as in table 3, example 1, one or more of the mixtures as in table 3, examples 2 to 11 are used.

### Example 24

### Face Cleansing Milk Type O/W

Phase A

| | |
|---|---|
| 1.50 g | Ceteareth-6 |
| 1.50 g | Ceteareth-25 |
| 2.00 g | Glyceryl Stearate |
| 2.00 g | Cetyl Alcohol |
| 10.00 g | Mineral Oil |

Phase B

| | |
|---|---|
| 5.00 g | Propylene Glycol |
| q.s. | Preservative |
| 1.00 g | of the mixture as in table 3, example 1 |
| 66.30 g | Aqua dem. |

Phase C

| | |
|---|---|
| 0.20 g | Carbomer |
| 10.00 g | Cetearyl Octonate |

Phase D

| | |
|---|---|
| 0.40 g | Tetrahydroxypropyl Ethylenediamine |

Phase E

| | |
|---|---|
| 0.10 g | Perfume oil/essential oil |
| 0.10 g | Bisabolol |

Good face cleansing milks are also obtained if, instead of the mixture as in table 3, example 1, one or more of the mixtures as in table 3, examples 2 to 11 are used.

### Example 25

### Transparent Soap

| | |
|---|---|
| 4.20 g | Sodium Hydroxide |
| 3.60 g | dist. water |
| 10.00 g | of the mixture as in table 3, example 1 |
| 22.60 g | Propylene Glycol |
| 18.70 g | Glycerol |
| 5.20 g | Cocoamide DEA |
| 2.40 g | Cocamine Oxide |
| 4.20 g | Sodium Lauryl Sulfate |
| 7.30 g | Myristic Acid |
| 16.60 g | Stearic Acid |
| 5.20 g | Tocopherol |

Good transparent soaps are also obtained if, instead of the mixture as in table 3, example 1, one or more of the mixtures as in table 3, examples 2 to 11 are used.

### Example 26

### Shaving Foam

| | |
|---|---|
| 6.00 g | Ceteareth-25 |
| 5.00 g | Poloxamer 407 |
| 52.00 g | Aqua dem. |
| 1.00 g | Triethanolamine |
| 5.00 g | Propylene Glycol |
| 1.00 g | PEG-75 Lanolin Oil |
| 5.00 g | of the mixture as in table 3, example 1 |
| q.s. | Preservative |
| 0.10 g | Perfume oil/essential oil |
| 25.00 g | Sodium Laureth Sulfate |

Bottling: 90 parts of active substance and 10 parts of propane/butane 25:75 mixture.

Good shaving foams are also obtained if, instead of the mixture as in table 3, example 1, one or more of the mixtures as in table 3, examples 2 to 11 are used.

### Example 27

### Aftershave Balm

Phase A

| | |
|---|---|
| 0.25 g | Acrylates/C10-30 Alkyl Acrylate Crosspolymer |
| 1.50 g | Tocopheryl Acetate |
| 0.20 g | Bisabolol |
| 10.00 g | Caprylic/Capric Triglyceride |
| q.s. | Perfume |
| 1.00 g | of the mixture as in table 3, example 1 |

Phase B

| | |
|---|---|
| 1.00 g | Panthenol |
| 15.00 g | Alcohol |
| 5.00 g | Glycerol |
| 0.05 g | Hydroxyethylcellulose |
| 1.90 g | of the mixture as in table 3, example 1 |
| 64.02 g | dist. water |

Phase C

| | |
|---|---|
| 0.08 g | Sodium Hydroxide |

Good aftershave balms are also obtained if, instead of the mixture as in table 3, example 1, one or more of the mixtures as in table 3, examples 2 to 11 are used.

### Example 28

### Care Cream

Phase A

| | |
|---|---|
| 2.00 g | Ceteareth-6 |
| 2.00 g | Ceteareth-25 |
| 2.00 g | Cetearyl Alcohol |
| 3.00 g | Glyceryl Stearate SE |
| 5.00 g | Mineral Oil |
| 4.00 g | Jojoba (Buxus Chinensis) Oil |
| 3.00 g | Cetearyl Octanoate |
| 1.00 g | Dimethicone |
| 3.00 g | Mineral oil, Lanolin Alcohol |

Phase B

| | |
|---|---|
| 5.00 g | Propylene Glycol |
| 0.50 g | Veegum |
| 1.00 g | Panthenol |
| 1.70 g | of the mixture as in table 3, example 1 |
| 6.00 g | Polyquaternium-44 |
| q.s. | Preservative |
| 60.80 g | Aqua dem. |

Phase C

| | |
|---|---|
| q.s. | Perfume |

Good care creams are also obtained if, instead of the mixture as in table 3, example 1, one or more of the mixtures as in table 3, examples 2 to 11 are used.

Oral and Dental Care Preparations

### Example 29

### Toothpaste

Phase A

| | |
|---|---|
| 34.79 g | Aqua dem. |
| 3.00 g | of the mixture as in table 3, example 1 |
| 20.00 g | Glycerol |
| 0.76 g | Sodium Monofluorophosphate |

Phase B

| | |
|---|---|
| 1.20 g | Sodium Carboxymethylcellulose |

Phase C

| | |
|---|---|
| 0.80 g | Aroma oil |
| 0.06 g | Saccharin |
| q.s. | Preservative |
| 0.05 g | Bisabolol |
| 1.00 g | Panthenol |
| 0.50 g | Tocopheryl Acetate |
| 2.80 g | Silica |
| 1.00 g | Sodium Lauryl Sulfate |
| 7.90 g | Dicalciumphosphate Anhydrate |
| 25.29 g | Dicalciumphosphate Dihydrate |
| 0.45 g | Titanium Dioxide |

Good toothpastes are also obtained if, instead of the mixture as in table 3, example 1, one or more of the mixtures as in table 3, examples 2 to 11 are used.

### Example 30

### Mouthwash

Phase A

| | |
|---|---|
| 2.00 g | Aroma oil |
| 4.50 g | of the mixture as in table 3, example 1 |
| 1.00 g | Bisabolol |
| 30.00 g | Alcohol |

Phase B

| | |
|---|---|
| 0.20 g | Saccharin |
| 5.00 g | Glycerol |
| q.s. | Preservative |
| 5.00 g | Poloxamer 407 |
| 52.30 g | Aqua dem. |

Good mouthwashes are also obtained if, instead of the mixture as in table 3, example 1, one or more of the mixtures as in table 3, examples 2 to 11 are used.

### Example 37

### Prosthesis Adhesive

Phase A

| | |
|---|---|
| 0.20 g | Bisabolol |
| 1.00 g | Beta-Carotene |
| q.s. | Aroma oil |
| 20.00 g | Cetearyl Octanoate |
| 5.00 g | Silica |
| 33.80 g | Mineral Oil |

Phase B

| | |
|---|---|
| 5.00 g | of the mixture as in table 3, example 1 |
| 35.00 g | PVP (20% strength solution in water) |

Good prosthesis adhesives are also obtained if, instead of the mixture as in table 3, example 1, one or more of the mixtures as in table 3, examples 2 to 11 are used.

### Example 38

### Liquid Soap

| | |
|---|---|
| 15.0 g | Coconut fatty acid, potassium salt |
| 3.0 g | Potassium oleate |
| 5.0 g | Luvitol ®Lite (BASF) |
| 2.0 g | Polymer vinylpyrrolidone/stearyl methacrylate |
| | 70/30% by weight (K value 47; 1% in isopropanol) |
| 1.0 g | Glycerol stearate |
| 0.5 g | of the mixture as in table 3, example 1 |
| 2.0 g | Ethylene glycol distearate |
| ad 100 | Specific additives, complexing agents, fragrances, water |

Good liquid soaps are also obtained if, instead of the mixture as in table 3, example 1, one or more of the mixtures as in table 3, examples 2 to 11 are used.

### Examples 39-41

### Conditioner Shampoo with Pearlescence

Data in % by weight

| Additive | Ex. 39 | Ex. 40 | Ex. 41 |
|---|---|---|---|
| Mixture as in table 3, example 1 | 0.5 | 0.5 | 0.5 |
| Sodium laureth sulfate | 9.0 | 9.0 | 9.0 |
| Cocoamidopropylbetaine | 2.5 | 2.5 | 2.5 |
| Benzophenone-3 | 1.5 | 0.5 | 1.00 |
| Pearlizing agent | 2.0 | 2.0 | 2.0 |
| Luvitol Lite ® (BASF) | 0.1 | 0.15 | 0.05 |
| Disodium EDTA | 0.1 | 0.2 | 0.15 |
| Preservative, perfume, thickener, pH | q.s. | q.s. | q.s. |
| adjustment and solubility promoter | | | |
| Water | ad 100.0 | ad 100.0 | ad 100.0 |

the pH is adjusted to 6.

Good conditioner shampoos with pearlescence are also obtained if, instead of the mixture as in table 3, example 1, one or more of the mixtures as in table 3, examples 2 to 11 are used.

### Examples 42-46

### Formulations for Showering, Washing, Bathing

Data in % by weight

| Additive | Ex. 42 | Ex. 43 | Ex. 44 | Ex. 45 | Ex. 46 |
|---|---|---|---|---|---|
| Texapon N 70 | 13.00 | 15.00 | 10.50 | 12.50 | 10.00 |
| Dehyton PK 45 | 7.50 | 7.00 | 5.00 | 5.50 | 10.00 |
| Cetiol HE | 2.00 | 2.50 | 3.50 | 5.00 | 2.30 |
| Perfume | 0.10 | 0.10 | 0.10 | 0.10 | 0.10 |
| Luvitol ® Lite (BASF) | 1.00 | 4.50 | 7.00 | 1.40 | 3.00 |
| D-Panthenol USP | 1.00 | 1.50 | 1.80 | 1.70 | 1.40 |
| Preservative | 0.10 | 0.10 | 0.10 | 0.10 | 0.10 |
| Citric acid | 0.10 | 0.10 | 0.10 | 0.10 | 0.10 |
| Mixture as in table 3, | | | | | |
| example 1 | 0.50 | 1.00 | 0.50 | 0.20 | 0.10 |
| Sodium chloride | 1.50 | 1.40 | 1.40 | 1.30 | 1.50 |
| Water dem. | ad 100 | ad 100 | ad 100 | ad 100 | ad 100 |

Good formulations for showering, washing, bathing are also obtained if, instead of the mixture as in table 3, example 1, one or more of the mixtures as in table 3, examples 2 to 11 are used.

The following examples are prepared, for example, as described in U.S. Pat. No. 6,451,300, columns 31 and 32.

| | Example |
|---|---|
| | 47 |
| | % by wt. |
| Ammonium laureth sulfate | 12 |
| Ammonium lauryl sulfate | 8 |
| Mixture as in table 3, example 1 | 0.4 |
| PEG-90M³ (INCI) | 0.5 |
| Zinc pyrithione⁴ (optional) | 1 |
| 1-Decene homopolymer⁵ | 0.3 |
| Trimethylpropane tricaprylate/caprate⁶ | 0.1 |
| Dimethicones⁷ (optional) | 2.0 |
| Ethylene glycol distearate | 2.0 |
| Cocamide MEA | 0.8 |
| Cetyl alcohol | 0.9 |
| Water and minor amounts | q.s. |

³PEG M_{w} ca. 4*10⁶ g/mol
⁴Zinc pyrithione with average particle size of ca. 2.5 μm;
⁵Puresyn 6 (hydrogenated polydecene)
⁶Mobil ®P43
⁷Visasil ® 330 000 cSt (General Electric Silicones).

Good shampoos are also obtained if, instead of the mixture as in table 3, example 1, one or more of the mixtures as in table 3, examples 2 to 11 are used.

Example

| | 48 | 49 |
|---|---|---|
| | % by wt. | % by wt. |
| Ammonium laureth sulfate | 12 | 10 |
| Ammonium lauryl sulfate | 6 | 6 |
| Polyquaternium-10 | — | 0.25 |
| Mixture as in table 3, example 1 | 0.25 | 0.25 |
| PEG-7M⁴ (INCI) | — | 0.1 |
| PEG-90M⁵ (INCI) | 0.1 | — |
| Zinc pyrithione⁶ (optional) | 1 | 1 |
| 1-Decene homopolymer⁷ | 0.4 | 0.4 |
| Trimethylpropanetricaprylate/caprate⁸ | — | 0.1 |
| Dimethicones⁹ (optional) | 1.15 | 1.35 |
| Ethylene glycol distearate | 1.0 | 1.5 |
| Cocamide MEA | 1.1 | 0.8 |
| Cetyl alcohol | 0.6 | 0.9 |
| Water and minor amounts | q.s. | q.s. |

⁴Polyox ®WSR N-750
⁵Polyox ®WSR N-301
⁶Zinc pyrithione with average particle size of ca. 2.5 μm;
⁷Puresyn 6 (hydrogenated polydecene)
⁸Mobil ®P43
⁹Visasil ® 330 000 cSt (General Electric Silicones)

Good shampoos are also obtained if, instead of the mixture as in table 3, example 1, one or more of the mixtures as in table 3, examples 2 to 11 are used.

| | Example |
|---|---|
| | 50 |
| | % by wt. |
| Sodium laureth sulfate | 10 |
| Sodium lauryl sulfate | 6 |
| Ethylene glycol distearate | 1.5 |
| Cocamide MEA | 0.8 |
| Cetyl alcohol | 0.9 |
| Mixture as in table 3, example 1 | 0.5 |
| Dimethicones⁹ (optional) | 2.35 |
| Trimethylpropanetricaprylate/caprate⁸ | 0.1 |
| 1-Decene homopolymer⁷ | 0.4 |
| Zinc pyrithione¹⁰ (optional) | 1.0 |
| Sodum citrate | 0.2 |
| Citric acid | 0.22 |
| Sodium chloride | 1.475 |
| Perfume | 0.7 |
| Sodium benzoate | 0.25 |
| Kathon ®CG | 0.0005 |
| Water | q.s. |

¹⁰Zinc pyrithione with average particle size of ca. 2.5 μm;
⁷Puresyn 6 (hydrogenated polydecene)
⁸Mobil ®P43
⁹Visasil ® 330 000 cSt (General Electric Silicones)

Good shampoos are also obtained if, instead of the mixture as in table 3, example 1, one or more of the mixtures as in table 3, examples 2 to 11 are used.

### Example 51

### Hair Repair Shampoo “Smooth & Silky”

| Phase | % by wt. | Ingredient | Source | INCI name |
|---|---|---|---|---|
| A | 0.5 | Mixture as in table 3, | | |
| | | example 1 | | |
| | 47.3 | Water, demineralized | | Aqua |
| B | 12.5 | Tego ®Betaine L7 | Evonik Goldschmidt | Cocamidopropyl betaine |
| | | | GmbH | |
| | 35.7 | Texapon ® NSO | Cognis Deutschland | Sodium laureth sulfate |
| | | | GmbH & Co. KG | |
| | 0.10 | Euxyl ® K 100 | Schülke & Mayr | Benzyl alcohol (and) |
| | | | GmbH | methyl chloroiso- |
| | | | | thiazolinone (and) |
| | | | | methyl chloroiso- |
| | | | | thiazolinone |
| | 0.30 | Perfume | | Fragrance |
| | 0.50 | D-Panthenol USP | BASF SE | Panthenol |
| | 0.10 | Edeta ® BD | BASF SE | Disodium EDTA |
| | 1.5 | NaCl | | Sodium chloride |

Preparation: add the components of phase B one after the other to the mixed phase A and stir until all of the components are completely dissolved. Properties: pH 6.5, viscosity: 9000 mPa·s (Brookfield DV II+sp. 4/20 rpm)

Good shampoos are also obtained if, instead of the mixture as in table 3, example 1, one or more of the mixtures as in table 3, examples 2 to 11 are used.

1. An aqueous or aqueous-alcoholic composition comprising
a) at least one cationic polymer a) and
b) at least one water-soluble polymer b) with a molecular weight M_{w} of at most 200 000 g/mol which comprises N-vinylimidazole in copolymerized form, where polymer a) consists of at least 50% by weight of copolymerized diallyldimethylammonium chloride and where the ratio of the amounts by weight of polymers a) and b) is in the range from 1:2 to 2:1.

2. The composition according to claim 1, where polymer a) consists of at least 90% by weight of copolymerized diallyldimethylammonium chloride.

3. The composition according to claim 1, where polymer b) consists of at least 10% by weight of copolymerized N-vinylimidazole.

4. The composition according to claim 1, where polymer b) has been prepared in the presence of at least one polyether-containing compound.

5. The composition according to claim 1, where the total amount of polymer a) and polymer h) is in the range from 0.001 to 20% by weight, based on the weight of the composition.

6. The composition according to claim 1 comprising at least one anionic surfactant as further ingredient c).

7. A method of producing aqueous or aqueous-alcoholic compositions according to claim 1, comprising
a) at least one cationic polymer a) and
b) at least one water-soluble polymer h) with a molecular weight M_{w} of at most 200 000 g/mol which comprises N-vinyl imidazole in copolymerized form, ingredients c) different from a) and b)
wherein
the polymers a) and b) are mixed together before anionic compounds are added to the composition.

8. A method of producing aqueous or aqueous-alcoholic compositions according claim 1, comprising
a) at least one cationic polymer a) and
b) at least one water-soluble polymer b) with a molecular weight M_{w} of at most 200 000 g/mol which comprises N-vinylimidazole in copolymerized form, wherein the polymers a) and b) are mixed together and monomers still present in the mixture are free-radically polymerized.

9. The method according to claim 7, wherein the mixing of the polymers a) and b) takes place at temperatures in the range from 10 to 100° C.

10. The use of at least one water-soluble polymer b) with a molecular weight M_{w} of at most 200 000 g/mol for stabilizing cosmetic preparations which comprise at least one cationic polymer a) as defined in claim 1 and at least one anionic surfactant.

11. The use of a composition according to claim 1 in cosmetic preparations for haircare and/or skincare.

12. A haircare or skincare agent comprising a composition according to claim 1.

13. The composition according to claim 2, where polymer b) consists of at least 10% by weight of copolymerized N-vinylimidazole.

14. The composition according to claim 2, where polymer b) has been prepared in the presence of at least one polyether-containing compound.

15. The composition according to claim 3, where polymer b) has been prepared in the presence of at least one polyether-containing compound.

16. The composition according to claim 2, where the total amount of polymer a) and polymer b) is in the range from 0.001 to 20% by weight, based on the weight of the composition.

17. The composition according to claim 3, where the total amount of polymer a) and polymer b) is in the range from 0.001 to 20% by weight, based on the weight of the composition.

18. The composition according to claim 4, where the total amount of polymer a) and polymer b) is in the range from 0.001 to 20% by weight, based on the weight of the composition.

19. The composition according to claim 2 comprising at least one anionic surfactant as further ingredient c).

20. The composition according to claim 3 comprising at least one anionic surfactant as further ingredient c).
